(19) United States
(12) Patent Application Publication
(10) Pub. No.: US 2002/0168639 A1
(43) Pub. Date: Nov. 14, 2002

(54) Profile array substrates

(76) Inventor: Patrick J. Muraca, Pittsfield, MA (US)

Correspondence Address: PALMER & DODGE, LLP, PAULA CAMPBELL EVANS, 111 HUNTINGTON AVENUE, BOSTON, MA, 02199, US

(21) Appl. No.: 09/781,021

(22) Filed: Feb. 9, 2001

Related U.S. Application Data
(60) Provisional application No. 60/234,493, filed on Sep. 22, 2000.

(51) Int. Cl. C12Q 1/68; G01N 33/574; G01N 1/30; G01N 33/48; C12M 1/34

(52) U.S. Cl. 435/6; 435/7.23; 435/40.5; 435/287.2

(57) ABSTRACT

The invention provides a profile array substrate comprising a first location for placing and retaining a test tissue sample, and a second location comprising a microarray. The microarray comprises a plurality of sublocations, each sublocation comprising a control tissue sample with known biological characteristics. Placement of the test tissue sample on the profile array substrate at the first location, permits a side-by-side comparison of the biological characteristics of a test tissue at the first location with the biological characteristics of tissues within the microarray.

### RELATED APPLICATIONS

This application claims priority under 35 U.S.C §119(e) to U.S. Provisional Application Serial No. 60/234,493, filed Sep. 22, 2000, the entirety of which is incorporated by reference herein.

### FIELD OF THE INVENTION

The invention relates to a profile array substrate comprising a first location for placement of a test tissue sample and a second location comprising a tissue microarray for comparison to the test tissue sample.

### BACKGROUND

Molecular medicine relies on the use of molecular probes to obtain diagnostic information about a patient. The Human Genome project has identified from 200,000 to 400,000 potential molecular probes, but the ability to determine the diagnostic relevance of these probes is hampered by the limits of current high throughput screening techniques with which to test a probe against a multiple patient types and tissue types.

Battifora, et al., in Lab Invest. 55: 244-248 (1986) and in U.S. Pat. No. 4,820,504, describe grouping multiple tissue specimens together on a single slide in order to screen hybridoma supernatants for antibodies. Formanski, et al., in U.S. Pat. No. 4,914,022, similarly describe a method for simultaneous histologic testing of multiple (24 to over 240) individual tissue specimens on a single slide, also as a means of screening and characterizing candidate monoclonal antibodies.

Kallioniemi, et al., in WO 99/44062 and WO 99/44062 disclose a method for rapid profiling of multiple tissue samples by incorporating the samples into a microarray on substrate such as a glass slide. The WO 99/44062 and WO 99/44062 applications describe a method of performing parallel analyses to identify molecular characteristics of tissues in the array. The results of analyses of tissues on a microarray are compared with the results of analyses of a test sample to determine if molecular characteristics of the test sample match those of a sample on the microarray.

### SUMMARY

The invention provides a profile array substrate to enable a side by side comparison of the biological characteristics of a test tissue with the biological characteristics of multiple control tissue samples. In one embodiment of the invention, a substrate for placement of a tissue sample is provided. The substrate comprises a first location on the substrate for placement of a tissue sample and a second location comprising a microarray. The microarray comprises a plurality of sublocations, each sublocation comprising a control tissue sample for which at least one biological characteristic is known.

In one embodiment, at least 50% of the sublocations of the microarray comprise different tissue types. In another embodiment, at least one tissue sample of the microarray is from a human. In a further embodiment, at least one tissue sample of the microarray is from a non-human mammal selected from the group consisting of a mouse, a rat, a rabbit, a ferret, a domestic animal, and a non-human primate. In still a further embodiment, at least one tissue sample comprises cells from a non-mammalian organism, selected from the group consisting of yeast, hydra, dictyostelium, Arabidopsis, a nematode, a fruit fly, a frog, a fish (e.g., such as a zebrafish), and a shark.

In one embodiment, the sublocations of the microarray on the profile array substrate comprise at least two tissue samples at different stages of development. In another embodiment, the sublocations of the microarray comprise cells from a genetically engineered organism, an organism comprising naturally arising mutations, or genetically engineered cells. In still embodiment, at least one tissue sample at a sublocation comprises cells from a knockout mouse. In a further embodiment, the knockout mouse comprises a disruption in at least one allele of the p53 gene, the MDM2 gene, or the VEGF gene. In still a further embodiment, two alleles are disrupted.

In one embodiment, the profile array substrate comprises a microarray comprising sublocations representing different doses of at least one expression product of a selected gene. In one embodiment, the selected gene is a tumor suppressor gene.

In another embodiment, at least one sublocation of the microarray comprises a cancer cell. In one embodiment, the tissue samples of the microarray represent different stages of cancer. In a further embodiment, the microarray substrate comprises at least one type of normal tissue (e.g., a non-cancerous tissue). In one embodiment, the microarray of the profile array substrate comprises from 2-1000 sublocations.

In one embodiment, the profile array substrate comprises glass; while in another embodiment, the substrate is selected from the group consisting of plastic, nylon, or nitrocellulose. In still another embodiment, the substrate is positively charged.

In one embodiment, the sublocations of the microarray comprise samples selected from the group consisting of frozen tissue, paraffin-embedded tissue, plastic-embedded tissue, cells from a bodily fluid, cells from a mucosal scraping, and cells from a tissue culture cell line. In another embodiment, the sublocations comprise any two of: frozen tissue, paraffin-embedded tissue, plastic-embedded tissues, cells from a bodily fluid, cells from a mucosal scraping, and cells from a tissue culture cell line.

In one embodiment, at least one sublocation comprises cells selected from the group consisting of brain cells, pituitary cells, eye cells, tongue cells, trachea cells, esophagus cells, heart cells, liver cells, spleen cells, muscle cells, lymph node cells, testis cells, cervix cells, uterus cells, placental cells, kidney cells, bladder cells, thyroid cells, adrenal gland cells, prostate cells, colon cells, uterine smooth muscle cells, tonsil cells, T-cells, hematopoietic progenitor cells, macrophages, pancreas cells, skin cells, adipose tissue cells, breast tissue cells, small intestine cells, rectum cells, stomach cells, salivary gland cells, and seminal vesicle cells.

In one embodiment, the microarray at the second location comprises at least one duplicate sublocation.

In one embodiment, the microarray comprises a plurality of sublocations comprising cells from individuals sharing a trait. In one embodiment, the shared trait is a genetic alteration, tumor size, gender, age, disease, predisposition to disease, kinship, death from the same disease, treatment with the same drug, exposure to chemotherapy, exposure to radiotherapy, exposure to hormone therapy, exposure to surgery, or exposure to the same environmental condition. In one embodiment, the shared trait is kinship, and each sublocation of the microarray comprises cells from different members of a pedigree. In another embodiment, the microarray further comprises environment-matched controls. In a further embodiment, the environment-matched controls include tissues from husbands, wives, children and/or stepparents.

In one embodiment, the microarray comprises a plurality of sublocations comprising cells from individuals sharing a plurality of traits. In another embodiment, each sublocation comprises a tissue sample from the same individual. In a further embodiment, at least one sublocation of the microarray includes cells which are suspected of being a target of a disease, such as cancer. In still a further embodiment, at least one sublocation comprises cells from a secondary metastasis.

In one embodiment, the microarray on the profile array substrate comprises at least one sublocation comprising cells from colorectal cancer tissue, prostate cancer tissue, breast cancer tissue, kidney cancer tissue, lung cancer tissue, urinary bladder cancer tissue, ovarian cancer tissue, brain tumor tissue, malignant melanoma tissue, or head and neck cancer tissue. In another embodiment, the microarray comprises at least two different types of cancerous tissue. In still another embodiment, at least twenty sublocations comprises cancerous tissues of the same tissue type from at least 20 genetically unrelated individuals. In a further embodiment, the tissue type is breast tissue, prostate tissue, ovarian tissue, or colon tissue. In still a further embodiment, the microarray comprises sublocations which comprise cells from cancerous tissue wherein the cells are selected from the group consisting of neoplastic cells, fibrous tissue cells, inflammatory tissue cells, apoptotic cells, normal cells, and combinations thereof.

In one embodiment, the microarray comprises sublocations including breast tissue comprising estrogen receptor positive and estrogen receptor negative cells. In another embodiment, the sublocations include breast tissue samples comprising progesterone receptor positive and progesterone receptor negative cells.

In one embodiment, tissue samples on the microarray are obtained from a cadaver. In another embodiment, sublocations of the microarray comprise tissue samples from a population of individuals. In one embodiment, the tissue samples are from individuals who have all died from the same disease. In another embodiment, the tissue samples are from individuals infected with HIV. In still a further embodiment, the population of individuals is a population of individuals having Tay Sachs disease, severe combined immunodeficiency disease, sickle cell anemia, or gulf war syndrome, or a predisposition to any of these diseases.

In one embodiment, the sublocations comprise tissues obtained from a tissue repository. In another embodiment, at least one sublocation comprises a tissue selected from the group consisting of tissue in a cancer tissue repository, tissue from a repository of tissues representing diseases affecting women, tissue from a repository of endocrine tissue specimens, tissue from a repository of cardiovascular tissue specimens, tissue from a repository of neurological tissue specimens, tissue from a repository of tissue specimens from individuals having an autoimmune disease, tissue from a repository of tissue specimens from individuals having a respiratory disease, and tissue from a repository of tissue specimens from individuals having an infectious disease.

In one embodiment, profile array substrate further comprises an identifier, such as a microchip for identifying the microarray on the substrate.

In one embodiment, any of the profile array substrates described above further comprise a test tissue sample at the first location.

The invention further provides recipient blocks for forming microarrays on a profile array substrate. In one embodiment, the recipient blocks are for forming any of the microarrays described above. In another embodiment, the recipient block comprises a frozen embedding media such as OCT™.

The invention also provides a method of evaluating a test tissue sample, comprising: providing a profile array substrate, placing a test tissue sample at the first location, reacting the test tissue and the microarray with a molecular probe, and comparing the reactivity of the test tissue with tissue samples in the microarray. In one embodiment, detection of the reaction of the molecular probe with a tissue sample identifies the expression of a gene. In another embodiment, the expression of the gene is the expresion of an RNA molecule and/or protein molecule. In a further embodiment, the molecular probe reacts with the modified form of a protein, but does not react with the unmodified form of the protein. In still a further embodiment, the reaction of the molecular probe with the tissue sample is correlated with the dosage of a gene in the tissue sample.

In one embodiment, the molecular probe is selected from the group consisting of a nucleic acid molecule, an antibody, an antigen binding portion of an antibody, an enzyme, a substrate for an enzyme, and combinations thereof. In one embodiment, the nucleic acid is a DNA molecule, an RNA molecule, or a PNA molecule. In one embodiment, the molecular probe is an antibody which is an an allele specific antibody.

In one embodiment, evaluating comprises determining the presence or absence of a pathology in the test tissue. In this embodiment, the microarray comprises at least one sublocation comprising a tissue sample having the pathology, and at least one sublocation comprising a tissue sample lacking the pathology. In one embodiment, the determining step is used to provide a diagnosis to an individual who is the source of the test tissue. In another embodiment of the invention, the method further comprises providing a report to the individual.

In one embodiment, the microarray on the profile array substrate used in the method comprises a plurality of tissue samples representing different stages of a progressive disease. In one embodiment, the progressive disease is cancer; while in another embodiment, the progressive disease is Parkinson's disease. In a further embodiment, evaluating the profile array substrate comprises determining the stage or absence of a pathology in the test tissue. In still a further embodiment, the step of determining is used to provide a prognosis to an individual who was the source of the test tissue. In another embodiment of the invention, the method further comprises providing a report to the individual.

In one embodiment, information relating to the reactivity of the test tissue is stored in a database. In one embodiment, the reactivity of tissue samples in the microarray with a molecular probe is known; while in another embodiment, the reactivity of tissue samples in the microarray with the molecular probe is unknown. In a further embodiment, information relating to the reactivity of tissue samples in the microarray is stored in a database.

In one embodiment, the method further comprises the step of providing access to the database. In another embodiment, the database is an electronic database. In a further embodiment, information relating to the reactivity of tissue samples in the microarray with at least one molecular probe is compared to information relating to the source of the tissues. In one embodiment, the information relating to the source of the tissues is patient information. In another embodiment, the patient information is any of: age, sex, weight, height, ethnic background, occupation, environment, family medical background, and the patient's medical history.

In one embodiment, the electronic database is coupled to an information management system which comprises a search function and a relationship determining function. In another embodiment, the tissue information management system identifies relationships between the biological characteristics of the test tissue and the tissues of the microarray. In a further embodiment, the relationship is the correlation between the expression of at least one biological characteristic and the presence or absence of a disease, or the stage of a disease. In one embodiment, the biological characteristic is the expression of an RNA molecule or a protein. In a further embodiment, the RNA molecule or protein is identified as a drug target, and the method further comprises the steps of identifying biomolecules which alter the biological activity of the RNA molecule or protein.

In one embodiment, the molecular probe comprises a panel of antibodies which is used to detect the presence or absence, or stage of, a pathology in a test tissue. In one embodiment, the pathology is selected from the group consisting of leukemia, lymphoma, Ewing's Sarcoma, rhabdomyosarcoma, and mesenchymal sarcoma.

In one embodiment of the invention, the method comprises evaluating a test tissue selected from the group consisting of breast tissue, prostate tissue, colon tissue, skin, lung tissue, and neural tissue. In another embodiment of the invention, evaluating comprises amplifying nucleic acids within the test tissue and the tissues in the microarray. In another embodiment, evaluating comprises performing immunohistochemistry on the test tissue and the tissues in the microarray. In still another embodiment, the method further comprises, after performing immunohistochemistry, isolating a portion of the test tissue sample and/or a tissue sample in the microarray and amplifying nucleic acids within the portion.

In one embodiment, the method of evaluating the test tissue further comprises the step of placing a profile array substrate in proximity to a light source and transmitting light from the light source to at least one tissue on the substrate. In another embodiment, the method further comprises providing a detector in proximity to the profile array substrate; the detector for detecting light transmitted from the tissue. In a further embodiment, the detector is in communication with a tissue information system comprising at least one user device connectable to the network which is in communication with an electronic database comprising information relating to said tissues on the microarray. In still a further embodiment, the user device is capable of displaying an image of the tissue on the display of the user device. In another embodiment, the user device is capable of displaying an interface on which information relating to the reactivity of said molecular probe can be entered for storage in the database.

The invention further provides kits for performing the method. In one embodiment, a kit is provided comprising any of: any of the profile array substrates described above and access to information relating to the sources of tissue on the microarrays included on these substrates. In one embodiment, the information comprises printed information. In another embodiment, the information comprises patient information. In a further embodiment, access is in the form of an identifier identifying the microarray and a password for accessing an electronic database in which tissue information is stored.

In one embodiment, the kit further comprises at least one molecular probe. In another embodiment, the kit comprises at least one profile array substrate comprising a plurality of different normal tissues. In a further embodiment, the kit comprises a profile array substrate which is labeled with an antibody which specifically recognizes a known antigen. In still a further embodiment, the kit additionally comprises the antibody which specifically recognizes the known antigen.

### BRIEF DESCRIPTION OF THE DRAWINGS

The objects and features of the invention can be better understood with reference to the following detailed description and accompanying drawings.

FIG. 1 is an illustration of a profile array substrate according to one embodiment of the invention, comprising six sublocations, each sublocation comprising cells from a different normal tissue type.

FIG. 2A is an illustration of a profile array substrate according to one embodiment of the invention, comprising a first location for placement of a test tissue sample and a second location comprising a microarray. The microarray comprises a plurality of sublocations; each sublocation representing a different stage of breast cancer.

FIG. 2B shows an array locator according to one embodiment of the invention for identifying coordinates on a microarray on a profile array substrate.

FIG. 2C shows six different tissue elements representing different stages of breast cancer stained with a CK7 antibody.

FIG. 2D shows information provided in a kit which comprises the profile array substrate shown in FIG. 2A and the array locator shown in FIG. 2B.

FIG. 2E shows a profile array substrate comprising a test tissue at a first location and a microarray at a second location. The test tissue and microarray are stained with a breast cancer-specific antibody.

### DESCRIPTION

The invention provides a profile array substrate comprising a first location for placing and retaining a test tissue sample and a second location comprising a microarray. The microarray comprises a plurality of sublocations, each sublocation comprising a tissue sample with at least one known biological characteristic. Placement of the test tissue sample on the profile array substrate at the first location permits a side-by-side comparison of the biological characteristics of a test tissue with the biological characteristics of tissues within the microarray.

### Definitions

In order to more clearly and concisely describe and point out the subject matter of the claimed invention, the following definitions are provided for specific terms which are used in the following written description and the appended claims.

As defined herein a “a sample” is a material suspected of comprising an analyte and includes a biological fluid, suspension, buffer, collection of cells, fragment or slice of tissue. A biological fluid includes blood, plasma, sputum, a pleural effusion, urine, cerebrospinal fluid, and leukophoresis samples.

As defined herein, “a tissue microarray” is a microarray that comprises a plurality of sublocations, each sublocation comprising tissue cells and/or extracellular materials from tissues, or cells typically infiltrating tissues, where the morphological features of the cells or extracellular materials at each sublocation are visible through microscopic examination. The term “microarray” implies no upper limit on the size of the tissue sample on the array, but merely encompasses a plurality of tissue samples which, in one embodiment, can be viewed using a microscope.

As defined herein, a “tissue” is an aggregate of cells that perform a particular function in an organism. The term “tissue” as used herein refers to cellular material from a particular physiological region. The cells in a particular tissue may comprise several different cell types. A non-limiting example of this would be brain tissue that further comprises neurons and glial cells, as well as capillary endothelial cells and blood cells. The term “tissue” also is intended to encompass a plurality of cells contained in a sublocation on the tissue microarray that may normally exist as independent or non-adherent cells in the organism, for example immune cells, or blood cells. The term is further intended to encompass cell lines and other sources of cellular material that now exist which represent specific tissue types (e.g., by virtue of expression of biomolecules characteristic of specific tissue types).

The term “like tissue” or the “same tissue type” or the refers to tissue that is of the same tissue type as a given tissue sample being investigated. For example, a sample of “like tissue” when considering a given prostate tissue means a sample of prostate tissue, from the same or a different individual. “Like tissue” can also include, for example, cultured or isolated cells from the same tissue type as a sample of tissue under investigation.

As used herein, an “identical tissue microarray” is a microarray obtained from the same recipient block which was the source of the microarray on the profile array substrate, and preferably from a section in the recipient block that was in proximity (i.e., less than 100 μm) to the section from which the microarray on the profile array substrate was generated. An “identical profile array substrate” refers to profile array substrates composing identical microarrays.

As defined herein, a “nucleic acid microarray,” a “peptide microarray” or “small molecule” microarray refers to a plurality of nucleic acids, peptides, or small molecules, respectively, that are immobilized on a substrate in assigned (i.e., known) locations on the substrate.

As defined herein, a “molecular probe” is any detectable molecule or molecule which produces a detectable molecule upon reacting with a biological molecule. “Reacting” encompasses binding, labeling, or catalyzing an enzymatic reaction. A “biological molecule” is any molecule which is found in a cell or within the body of an organsim.

As defined herein, a “probe which identifies the expression of a gene” is a probe whose reaction with a tissue sample indicates that the gene is expressed in the tissue sample.

As used herein, the term “detectable binding reagent” refers to an agent that specifically recognizes and interacts or binds with an entity one wishes to measure, wherein the agent has a property permitting detection when bound. “Specifically interact” means that a binding agent physically interacts with the entity one wishes to measure, to the exclusion of other entities also present in the sample. The binding of a detectable binding reagent useful according to the invention has stability permitting the measurement of the binding. A detectable binding reagent can possess an intrinsic property that permits direct detection (“direct labeling”), or it can be labeled with a detectable moiety for subsequent detection or reacted with a substrate to produce detectable products (“indirect labeling”).

As used herein, the term “detectable moiety” refers to a moiety that can be attached to a binding reagent that confers detection of the binding reagent by a particular method or methods. Detectable moieties include, but are not limited to, radiolabels (e.g., ³²P, ³⁵S, ¹²⁵I, etc.), enzymes (e.g., alkaline phosphatase, peroxidase, etc.), fluorophores (e.g., fluorescein, amino coumarin acetic acid, tetramethylrhodamine isothiocyanate (TRITC), Texas Red, Cy3.0, Cy5.0, green fluorescent protein, etc.) and colloidal metal particles.

As used herein, the term “labeled” means that a detectable moiety has been physically attached to a binding reagent.

As used herein, the term “antibody or antigen binding fragment thereof” or “antigen binding portion” refers to an immunoglobulin or a fragment or portion thereof, having the capacity to specifically bind a given antigen. The term “antibody” as used herein is intended to include whole antibodies of any isotype (IgG, IgA, IgM, IgE, etc), and fragments thereof which are also specifically reactive with a antigen. Antibodies can be fragmented using conventional techniques and the fragments or portions screened for specific binding in the same manner as whole antibodies. Thus, the term includes fragments of proteolytically-cleaved or recombinantly-prepared portions of an antibody molecule that are capable of selectively reacting with a certain antigen. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's can be covalently or non-covalently linked to form antibodies having two or more binding sites. Antibodies can be labeled with detectable moieties by one of skill in the art. In some embodiments, the antibody that binds to an entity one wishes to measure (i.e., the primary antibody) is not labeled, but is instead detected by binding of a labeled secondary antibody that specifically binds to the primary antibody.

As used herein, “antibody staining” or “staining by an antibody” refers to the binding of an antibody to an antigen which is detected, either through direct or indirect labeling.

As defined herein, “an individual” is a single organism and includes humans, animals, plants and protists.

As used herein, the term “biological characteristics of a tissue” refers to the phenotype and genotype of the tissue or cells within a tissue, and includes characteristics such as tissue type, morphological features, the expression of biological molecules within a tissue (e.g., such as the expression and accumulation of RNA molecules, the expression and accumulation of proteins, including the expression of their modified, cleaved, or processed forms, and further including the expression and accumulation of enzymes, their substrates, products, and intermediates), and the expression and accumulation of metabolites, carbohydrates, lipids, and the like). A biological characteristic can also be the ability of a tissue to bind, incorporate, or respond to a drug or agent. As used herein, “biological characteristics of a tissue source” are the characteristics of the organism which is the source of the tissue on the microarray (e.g., such as the age, sex, and physiological state of the organism).

As used herein, the term “information about a patient” refers to any information known about the individual from whom a tissue sample was obtained (including information about biological characteristics). The term “patient” does not necessarily imply that the individual has ever been hospitalized or received medical treatment prior to obtaining a tissue sample. The term “patient information” includes, but is not limited to, age, sex, weight, height, ethnic background, occupation, environment, family medical background, the patient's own medical history (e.g., information pertaining to prior diseases, diagnostic and prognostic test results, drug exposure or exposure to other therapeutic agents, responses to drug exposure or exposure to other therapeutic agents, results of treatment regimens, their success, or failure, history of alcoholism, drug or tobacco use, cause of death, and the like). The term “patient information” refers to information about a single individual; information from multiple patients provides “demographic information,” defined as statistical information relating to populations of patients, organized by geographic area or other selection criteria. Demographic information can include “epidemiological information,” defined as information relating to the incidence of disease in populations.

As defined herein, the term “information relating to” is information which summarizes, reports, provides an account of, and/or communicates particular facts, and in some embodiments, includes information as to how facts were obtained and/or analyzed.

As used herein, the term, “in communication with” refers to the ability of a system or component of a system to receive input data from another system or component of a system and to provide an output in response to the input data. “Output” may be in the form of data or may be in the form of an action taken by the system or component of the system.

As defined herein, “a diagnostic trait” is an identifying characteristic, or set of characteristics, which in totality are diagnostic. The term “trait” encompasses both biological characteristics and experiences (e.g., such as exposure to a drug, occupation, place of residence). In one embodiment, a trait is a marker for a particular cell type, such as a transformed, immortalized, pre-cancerous, or cancerous cell, or a state (e.g., a disease) and detection of the trait provides a reliable indicia that the sample comprises that cell type or state. Screening for an agent affecting a trait thus refers to identifying an agent which can cause a detectable change or response in a trait which is statistically significant (i.e., within 95% confidence levels).

A “disease” or “pathology” is a change in one or more biological characteristics that impairs normal functioning of a cell, tissue, and/or organism.

As defined herein, “a cell proliferative disorder” is a condition marked by any abnormal or aberrant increase in the number of cells of a given type or in a given tissue. Cancer is often thought of as the prototypical cell proliferative disorder, yet disorders such as atherosclerosis, restenosis, psoriasis, inflammatory disorders, some autoimmune disorders (e.g., rheumatoid arthritis) are also caused by abnormal proliferation of cells, and are thus also examples of cell proliferative disorders.

As used herein, the term “course of disease” refers to the sequence of events in which a disease develops, causes symptoms, and is either recovered from, or continues, and/or increases in severity.

As used herein, the term “cancer” refers to a malignant disease caused or characterized by the proliferation of cells which have lost susceptibility to normal growth control. The term “cancer cells” encompasses the term “tumor cells” or “malignant cells” or neoplastic cells.” “Malignant disease” refers to a disease caused by cells that have gained the ability to invade either the tissue of origin or to travel to sites removed from the tissue of origin. The term “cancer cells” encompasses cells which form a tumor.

As defined herein, “a tumor” is a neoplasm that may either be malignant or non-malignant. Tumors of the same tissue type originate in the same tissue, and may be divided into different subtypes based on their biological characteristics.

As used herein, the term “tumor stage” or “tumor grade” or “cancer stage” or “cancer grade” refers to a measure of the degree of advancement or progression of a tumor. A tumor's stage is determined according to criteria including, for example, the morphology of the cells, morphology of the tissue in which the cells are found, whether tumor cells have infiltrated the tissue of origin, whether tumor cells have invaded lymph nodes, and whether distant metastasis has occurred. Clinical staging for many tumors follows the TNM system, but other clinical staging scales adapted to specific diseases are known in the art (see, e.g., as defined in U.S. Provisional Application Serial No. ______, “Oncology Tissue Microarrays,” filed Sep. 29, 2000 (Attorney Docket No. 5568/1040), the entirety of which is incorporated by reference herein.

As used herein, the term “degree of disease severity” refers to measure of how advanced a disease is, on a scale from no disease to the worst possible disease. One of skill in the art can place a set of tissue samples representing a disease in order of ascending or descending severity of disease on the microarrays of the invention. In order to do so, samples may be compared not only to known standards, but also to each other.

As used herein, the term “control tissue” is a tissue for which at least one biological characteristic is known and which provides a standard against which to measure the expression of that biological characteristic in a test tissue.

As used herein, the term “difference in biological characteristics” refers to an increase or decrease in a measurable expression of a given biological characteristic. A difference may be an increase or a decrease in a quantitative measure (e.g., amount of a protein or RNA encoding the protein) or a change in a qualitative measure (e.g., location of the protein). Where a difference is observed in a quantitative measure, a “significant difference” according to the invention will be at least 10% greater or less than the level in a normal standard sample. Where a difference is an increase, the increase may be as much as 20%, 30%, 50%, 70%, 90%, 100% (2-fold) or more, up to and including 5-fold, 10-fold, 20-fold, 50-fold or more. Where a difference is a decrease, the decrease may be as much as 20%, 30%, 50%, 70%, 90%, 95%, 98%, 99% or even up to and including 100% (no specific protein or RNA present). It should be noted that even qualitative differences may be represented in quantitative terms if desired. For example, a change in the intracellular localization of a polypeptide may be represented as a change in the percentage of cells showing the original localization.

As used herein, the term “expression status” refers to the amount or presence of a measurable quality of a biological characteristic describing an aspect of its amount or presence in a tissue. Expression status includes, for example, the total level or relative level of expression of a biological characteristic, the percentage of cells that express a biological characteristic in a tissue, the localization of a biological characteristic in the cells, the conformation of a biological characteristic, the presence, absence, or degree of modifications of a biological characteristic (e.g., post-translational modification of a polypeptide, association or lack of association with co-factors or regulatory subunits, methylation or the presence of adducts or intramolecular cross-links within a nucleic acid, or the degree or type of cross-linking of a carbohydrate), or the activity of a biological characteristic (e.g., an enzyme or positive or negative regulatory factors of an enzyme). Expression status also includes the presence, absence or proportion of cells having a particular nuclear or cellular morphology.

As defined herein, a “population” refers to a group of individuals sharing a common biological characteristic or inhabiting a particular geographic locality. A population comprises a number of individuals large enough to provide a statistical measurement.

As used herein, “correlation of disease status” refers to a structural, functional, or qualitative correspondence between at least two comparable entities that relate the status of an individual's disease to the disease status of another individual or group of individuals. As used herein, the term “disease status” refers to the stage or grade of a disease, especially a cell proliferative disease.

As used herein, the term “cell growth-related biological characteristic” refers to a characteristic that is involved in, or correlates with, the pathogenesis of a cell proliferative disease. Such characteristics can act in a positive or negative manner, as long as some aspect of their expression influences or correlates with the presence or progression of a cell proliferative disease.

As used herein, the term “a normal tissue sample” refers to a tissue sample which does not express morphological or biomolecular markers of disease and which can perform normal physiological functions. As used herein, “a non-cancerous tissue” is a tissue which does not express the morphological or biomolecular markers of cancer and which proliferates in a normal manner.

As used herein, “a cancer-specific marker” or a “tumor specific antigen” is a biomolecule which is expressed preferentially on cancer cells/tumor cells and is not expressed, or is expressed to small degree, in non-cancer non-tumor cells of an adult individual. As used herein, “a small degree” means that the difference in expression of the marker in cancer cells and non-cancer cells is large enough to be detected as a statistically significant difference when using routine statistical methods to within 95% confidence levels. A cancer-specific marker is any biomolecule that is involved in, or correlates with, the pathogenesis of a cell proliferative disease, and can act in a positive or negative manner, as long some aspect of its expression or form influences or correlates with the presence or progression of a cell proliferative disease. While in one embodiment, expressed levels of a biomolecule provide an indicia of cancer progression or reoccurrence, in another embodiment of the invention, the expressed form of a biomolecule provides the indicia (e.g., a cleaved or uncleaved state, a phosphorylated or unphosphorylated state).

As used herein, a “breast cancer-specific marker” as distinguished from a “cancer-specific marker” is a biomolecule that specifically correlates with the pathogenesis of breast cancer tissues but is not found, or is found at significantly lower levels, in other types of cancerous tissues.

As used herein, the term “disease recurrence” refers to the development or emergence of cells of a proliferative disease, such as a tumor, after a treatment that has substantially removed such cells. A disease recurrence may be at the same site as the original disease or elsewhere, but will involve abnormal proliferation of cells of the same tissue of origin as in the original disease.

As defined herein, the “efficacy of a drug” or the “efficacy of a therapeutic agent” is defined as ability of the drug or therapeutic agent to restore the expression status of a diagnostic trait to values not significantly different from normal (as determined by routine statistical methods, to within 95% confidence levels).

As used herein, a “tissue information retrieval database” or “database” is a collection of information or facts which is cross-referenced, or indexed, with tissues on a microaray. A database can include paper or audio information as well as electronic records.

As defined herein, an “electronic database” is a collection of information or facts stored in the memory of a computer (e.g., such as a server or host computer) and organized according to a data model which determines whether the data is ordered using linked files, hierarchically, according to relational tables, or according to some other model determined by a system operator. The organization scheme that the electronic database uses is not critical, so long as information within the electronic database is accessible to the user through an information management system. Data in the electronic database are stored in a format consistent with an interpretation based on definitions established by the system operator (i.e., the system operator determines the fields which are used to define patient information, molecular profiling information, or another type of information category).

As defined herein, “a system operator” is an individual who controls access to the electronic database.

As used herein, the term “information management system” refers to a system which comprises a plurality of functions for accessing and managing information within the electronic database Minimally, an information management system according to the invention comprises a search function, for locating information within the database and for displaying a least a portion of this information to a user, and a relationship-determining function, for identifying relationships between information or facts stored in the database.

As defined herein, an “interface” or “user interface” or “graphical user interface” is a display comprising text and/or graphical information displayed by the screen or monitor of a user device connectable to the network, which enables a user to interact with the electronic database and information management system.

The term “providing access to at least a portion of a database” as defined herein refers to making information in the database available to user(s) through a visual or auditory means of communication.

As used herein, “through a visual means of communication” includes displaying or providing written text, image(s), sign language, or a combination of written and graphical information to a user of the database. Written and/or graphical information can be communicated through a printed report or electronically (e.g., through a display on the display of a computer or other processor, through email or other electronic messaging systems, through a wireless communication device, via facsimile, and the like).

As used herein, “through an auditory means of communication” refers to providing the user with taped audio information, or access to another user who can communication the information through speech.

The term “report” as used herein refers to a record or summary of the information which may be provided in written, graphical, electronic, or audio form, or combinations of these forms, as described above.

“High throughput techniques” are techniques that evaluate large numbers (at least 10) of samples at a single time.

As used herein, “molecular procedure” refers to contact with a test reagent or molecular probe, such as an antibody, nucleic acid probe, enzyme, chromagen, label, and the like, and/or to buffers, solvents, and temperature conditions routinely used in molecular detection assays such as immunohistochemistry, nucleic acid hybridization (including PCR, RT-PCR, in situ hybridization, and the like), and staining reactions (e.g., such as reactions which use hematoxylin and eosin or other dyes).

The Profile Array Substrate

The profile array substrate facilitates handling of both a microarray and test tissue sample during a variety of molecular procedures, enabling a side by side comparison of biological characteristics detected in a test tissue with biological characteristics detected in tissues in the microarray. In one embodiment, the molecular procedure comprises a plurality of hybridizations, incubations, fixation steps, dehydration steps, changes of temperature (from −4° C. to 100° C.), exposures to solvents, and/or wash steps.

In one embodiment of the invention, the profile array substrate is transparent and solvent-resistant. In this embodiment, the profile array substrate comprises any of: glass, quartz, fused silica, or other nonporous substrate, plastic, such as polyolefins, polyamides, polyacarylamides, polyesters, polyacrylic esters, polycarbonates, polytetrafluoroethylene, polyvinyl acetates, and plastic compositions containing fillers (such as glass fillers), extenders, stabilizers, and/or antioxidants; celluloid, cellophane or urea formaldehyde resins, or other synthetic resins such as cellulose acetate ethylcellulose, or other transparent polymer.

In one embodiment, the profile array substrate is rigid; however, in another embodiment, the profile array substrate is semi-rigid or flexible (e.g., the substrate is a flexible plastic comprising polycarbonate, cellular acetate, polyvinyl chloride, and the like). In the latter embodiment, a portion of the profile array substrate comprising the first location can be touched directly to a sample, e.g., by folding the substrate.

In another embodiment, the profile array substrate is optimized for use in conjunction with an optical system. For example, in one embodiment, the profile array substrate is optically opaque and substantially non-fluorescent (e.g., for use in applications where fluorescent labels are used to identify or confirm biological characteristics). Nylon or nitrocellulose membranes can also be used as profile array substrates and include materials such as polycarbonate, polyvinylidene fluoride (PVDF), polysulfone, mixed esters of cellulose and nitrocellulose, and the like.

The size and shape of the profile array substrate may generally be varied. However, preferably, the substrate fits entirely on the stage of a microscope. In one embodiment, the profile array substrate is planar; however, in another embodiment, the substrate comprises irregularities or cavities. In another embodiment of the invention, at least the second location of the profile array substrate comprising the microarray is entirely planar. In one embodiment of the invention, the profile array substrate is 1 inch by 3 inches, 77×50 mm, or 22×50 mm.

The First Location (For Receiving a Test Tissue)

In one embodiment of the invention, the first location is a portion of the substrate which can form a stable association with a test tissue specimen, such that the test tissue specimen retains its position on the substrate during at least one molecular procedure. In one embodiment of the invention, the test tissue is a tissue section (e.g., a section of a frozen, paraffin-, or plastic-embedded tissue block). However, in another embodiment, the tissue is a sample of cells from a bodily fluid (e.g., blood, plasma, lymph, CSF fluid, a pleural effusion, or a leukophoresis sample), the cells of which have been fixed and squashed, or flattened, upon the substrate, or, alternatively, snap frozen on the substrate. In a still further embodiment, the cells are obtained from a mucosal scraping (e.g., from an oral or vaginal cavity). In a preferred embodiment of the invention, the test tissue is a tissue which has morphological features substantially intact and which can be at least viewed under a microscope to distinguish subcellular features (e.g., such as a nucleus, an intact cell membrane, organelles, and/or other cytological features), i.e., the tissue is not lysed.

In one embodiment of the invention, the first location for receiving the test tissue comprises an agent for enhancing test tissue retention. Agents encompassed within the scope of the invention, include, but are not limited to, a plurality of positively charged molecules (e.g., poly-lysine), gelatin, an adhesive, and Vectabond™ (Vector Laboratories).

The size of the first location can vary. In one embodiment of the invention, the first location is at least 2-20 mm×2-20 mm, or 10-200 mm×10-200 mm. In one embodiment, the first location is at least 20 mm×22 mm, at least 22 mm×22 mm, at least 22 mm×30 mm, at least 22 mm×40 mm, at least 22 mm×50 mm, at least 22 mm×60 mm, at least 22 mm×70 mm, at least 22 mm×80 mm, at least 22 mm×90 mm, or at least 22 mm×100 mm.

The Second Location Comprising a Microarray

The second location comprises a microarray, the microarray comprising a plurality of sublocations. Each sublocation comprises a control tissue stably associated therewith (e.g., able to retain its position relative to another sublocation and relative to the first location after exposure to at least one molecular procedure). In one embodiment, the control tissue at each sublocation is a tissue which has morphological features substantially intact and which can be at least viewed under a microscope to distinguish subcellular features (e.g., such as a nucleus, an intact cell membrane, organells, and/or other cytological features), i.e., the tissue is not lysed.

In one embodiment of the invention, the microarray comprises from 2-1000 sublocations. In another embodiment, the microarray comprises 2 sublocations, 5 sublocations, 10 sublocations, 20 sublocations, 25 sublocations, 30 sublocations, 45 sublocations, 50 sublocations, 55 sublocations, 60 sublocations, 65 sublocations, 75 sublocations, 100 sublocations, 150 sublocations, 200 sublocations, 250 sublocations, or 500 sublocations, 550 sublocations, 600 sublocations, 650 sublocations, 700 sublocations, 750 sublocations, 800 sublocations, 850 sublocations, 900 sublocations, 950 sublocations, or 1000 sublocations. In one embodiment of the invention, each sublocation is from 2-10 mm apart. In another embodiment of the invention, each sublocation comprises at least one dimension which is 600 μm-20 mm. The sublocations can be organized in any pattern, and each sublocation can be generally any shape (square, circular, oval, elliptical, disc shaped, rectangular, triangular, and the like).

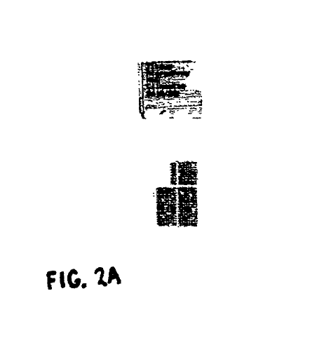

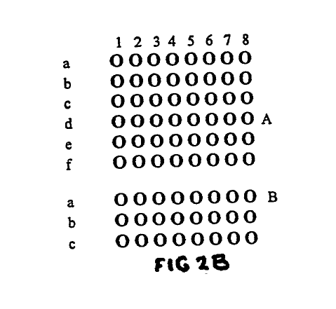

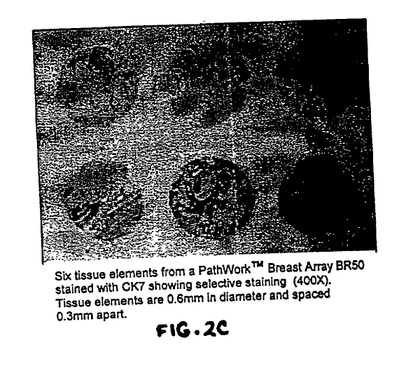

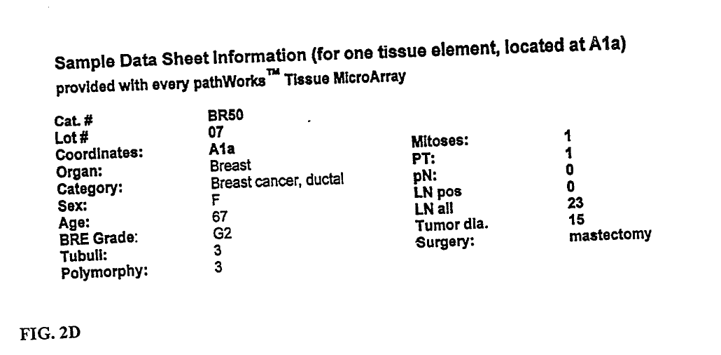

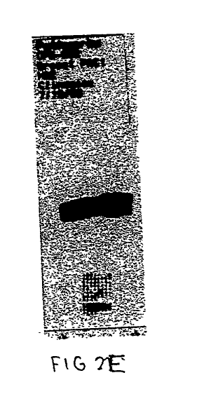

In a preferred embodiment, the sublocations are positioned in a regular repeating pattern (e.g., rows and columns) such that the identification of each sublocation (e.g., as to tissue type, and source) can be ascertained by the use of an array locator (as shown in FIG. 2B). In one embodiment, the array locator is a template having a plurality of shapes, each shape corresponding to the shape of each sublocation in the microarray, and maintaining the same relationships as each sublocation on the micorarray. The array locator is marked by coordinates, allowing the user to readily identify a sublocation on the micorarray by virtue of unique coordinates. In one embodiment of the invention, the array locator is a transparent sheet (e.g., plastic, acetate, and the like). In another embodiment of the invention, the array locator is a sheet comprising a plurality of holes, each hole corresponding in shape and location to each sublocation on the micorarray.

The second location comprising the microarray can be located anywhere on the profile array substrate, so long as there is a first location of sufficient size to accommodate an at least 2-20 mm tissue section (i.e., the section can lay flatly on the first location substrate without crumpling). In one embodiment, the second location is of sufficient size to accommodate a first location comprising an at least 2 mm tissue, an at least 6 mm tissue, an at least 10 mm tissue, or an at least 20 mm test tissue. In a preferred embodiment, the profile array substrate comprises a first and a second end (e.g., such as an end of a glass slide) and the second location is proximal to one of the ends. In another embodiment of the invention, at least one boundary of the second location corresponds with an edge of the substrate. In a further embodiment of the invention, the midpoint of the second location (whose boundaries are defined by the boundaries of the microarray) does not coincide with the midpoint of the substrate.

Sources of Tissue

Tissues may be obtained from a variety of sources. In one embodiment, cells are obtained from cadavers or preserved dead organisms, e.g., from blocks of a solid or semi-solid embedding material which preserves a tissue's morphology (e.g., paraffin, a frozen tissue embedding medium, such as OCT, and the like). In a further embodiment of the invention, cells are obtained from biopsies from a living organism, or other surgical or pathology procedures. In still a further embodiment of the invention, the cells are from cell lines, or primary cell cultures.

In one embodiment of the invention, at least one sublocation comprises any of: cells from the brain, pituitary, eye, tongue, trachea, espophagus, heart, liver, spleen, muscle, lymph node, testis, cervix, uterus, placenta, kidney, bladder, thyroid, adrenal gland, prostate, colon, uterine smooth muscle, tonsil, groups of T-cells, hematopoietic progenitor cells, macrophages, pancreas, skin, adipose tissue, breast tissue, small intestine, rectum, stomach, salivary glands, and seminal vesicles.

In one embodiment of the invention, each sublocation comprises the same tissue type, to form a brain array, pituitary array, eye array, tongue array, trachea array, espophagus array, heart array, liver array, spleen array, muscle array, lymph node array, testis array, cervix array, uterus array, placenta array, kidney array, bladder array, thyroid array, adrenal gland array, prostate array, colon array, uterine smooth muscle array, tonsil array, T-cell array, hematopoietic progenitor cell array, macrophage array, pancreas array, skin array, adipose tissue array, breast tissue array, small intestine array, rectum array, stomach array, salivary gland array, or seminal vesicle array.

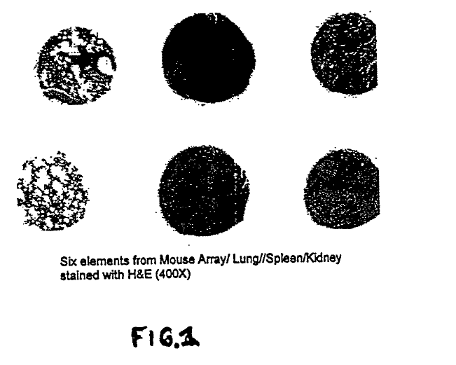

However, in another embodiment of the invention, the microarray at least two sublocations comprising cells from different tissues. In one embodiment of the invention, at least 50% of the sublocations in the microarray comprise cells from different tissues. In still a further embodiment of the invention, at least 60%, 70%, 80%, 90%, or 100% of the array comprises cells from different tissues. For example, FIG. 1 is an illustration of a profile array substrate according to one embodiment of the invention, comprising six sublocations, each sublocation comprising cells from a different normal tissue type (i.e., mouse tissue).

In a further embodiment of the invention, the microarray comprises at least one duplicate sublocation (e.g., a sublocation comprising cells from like tissue, and/or from the same representative area on a tissue). In one embodiment of the invention, the microarray comprises at least 5 duplicate sublocations, at least 10 duplicate sublocations, or at least 20 duplicate sublocations. Duplicate sublocations can comprise duplicates of normal or disease tissue samples (e.g., such as tissue samples comprising abnormally proliferating cells).

In one embodiment of the invention, the microarray comprises a plurality of sublocations comprising cells from individuals sharing a trait. In one embodiment of the invention, the trait shared is tumor diameter, gender, age, a disease, predisposition to a disease, kinship, death from the same disease, treatment with the same drug, exposure to chemotherapy or radiotherapy, exposure to hormone therapy, exposure to surgery, exposure to the same environmental condition (e.g., such as exposure to carcinogens, pollutants, asbestos, TCE, perc., benzene, chloroform, nicotine and the like), the same genetic alteration, or group of genetic alterations, expression of the same gene or sets of genes, the same species, breed, or strain (e.g., in the case of a non-human individual).

In one embodiment of the invention, the trait is kinship and each sublocation of the microarray comprises cells from different members of a pedigree (e.g., selected from the group consisting of sibs, twins, cousins, mothers, fathers, grandmothers, grandfathers, uncles, aunts, and the like). In another embodiment of the invention, the “pedigree microarray” comprises environment-matched controls (e.g., tissues from husbands, wives, adopted children, stepparents, and the like). In still a further embodiment of the invention, the microarray is a reflection of a plurality of traits representing a particular patient demographic group of interest, e.g., overweight smokers, diabetics with peripheral vascular disease, individuals having a particular predisposition to disease (e.g., sickle cell anemia, Tay Sachs, severe combined immunodeficiency, and the like).

In another embodiment of the invention, the trait is a disease or a predisposition to a disease (e.g., from genetic alteration(s), from an infectious agent, and/or from exposure to an environmental condition). In one embodiment of the invention, the disease comprises a blood disorder, blood lipid disease, autoimmune disease, bone or joint disorder, cancer (including, but not limited to, breast ductal carcinoma, bladder carcinoma, leiomyoma, meningioma. melanoma, melanoma with a Clark score of 1-5 with nevus, seminoma, lymphoma, and colon adenocarcinoma), cancer susceptibility, a cardiovascular disorder, a respiratory disease, an endocrine disorder, an immune disorder, an infectious disease, a muscle wasting or whole body wasting disorder, a neurological disorder, a skin disorder, a kidney disease (e.g., such as caused by excessive fibrosis), scleroderma, stroke, hereditary hemorrhage telangiectasia, a disorder associated with diabetes, such as hypertension, retinal neuropathy, peripheral vascular disease, and the like, Gaucher disease, cystic fibrosis and sickle cell anemia, or a psychiatric disorder including, but not limited to, manic depression or bipolar disorder, depression, borderline personality disorder, anxiety, schizophrenia, and the like. For further discussion of human genetic diseases, see “Mendelian Inheritance in Man: A Catalog of Human Genes and Genetic Disorders” by Victor A. McKusick (12th Edition (3 volume set) June 1998, Johns Hopkins University Press, ISBN: 0801857422), the entirety of which is incorporated by reference herein.

In one embodiment, the cells at a particular sublocation are from an organ or tissue suspected to be a target of the disease: e.g., the brain, in the case of an individual with schizophrenia, the eye, in the case of an individual with retinoblastoma, hematopoeitic progenitor cells, in the case of an individual with leukemia, lungs in the case of an individual with exposure to asbestos. However, in another embodiment, the microarray comprises a plurality of tissues, including the target tissue; i.e., providing a “system array” which is representative of the body of an individual (or population of individuals). In a further embodiment of the invention, the cells are from cadavers. In still a further embodiment of the invention, the cells are from individuals who have all died from the same pathology, or from individuals being treated with the same drug (including those who recovered from the disease and/or those who did not). In another embodiment, the cells are from a cell culture (either primary or continuous).

In one embodiment of the invention, the second location comprises a tissue microarray comprising a plurality of normal tissues for comparison to diseased tissues either on the microarray itself or which form part of the test tissue sample provided at the first location. In one embodiment of the invention, normal tissues are selected from the group consisting of cerebrum, cerebellum, heart, lung, thyroid gland, adrenal gland, skin, parotis, pancreas, stomach (corpus), stomach (antrum), small intesting, colon, liver, gall bladder, tonsil, spleen, lymph node, endometrium, (proliferation), endometrium(secretion), placenta (last trimenon), placenta (first trimenon), kidney, prostate, testis, epidydimis, skeletal muscle, smooth muscle. In one embodiment, a microarray is provided comprising all of these tissues. In another embodiment of the invention, the microarray further comprises duplicates of each normal tissue sample.

In one embodiment of the invention, a profile array substrate is provided comprising a microarray which comprises a plurality of different types of cancerous tissues and a plurality of different normal tissues, such that for each cancerous tissue sublocation, there is a normal tissue sublocation corresponding to the tissue type of origin of the cancerous tissue (e.g., where a sublocation comprises breast tumor tissue, another sublocation is provided comprising normal breast tissue).

In one embodiment, the microarray comprises at least one sublocation comprising cells from colorectal cancer tissue, prostate cancer tissue, breast cancer tissue, kidney cancer tissue, lung cancer tissue, urinary bladder cancer tissue, ovarian cancer tissue, brain tumor tissue, malignant melanoma tissue, and head and neck cancer tissue. In one embodiment, a single type of cancerous tissue is represented on the microarray (with, or without, sites of secondary metastases). However, in another embodiment, a plurality of different cancer types or tumor types are represented on a single microarray. In one embodiment, at least 20 sublocations comprise cancerous tissues of the same tissue type from at least 20 genetically unrelated individuals. In another embodiment of the invention, a set of profile array substrates is provided representing more than 30,000 different tumor types.

In one embodiment, sublocations comprise cells from cancerous tissue which are selected from the group consisting of: neoplastic cells, fibrous tissue cells, inflammatory tissue cells, necrotic cells, apoptotic cells, normal cells, and combinations thereof. In another embodiment of the invention, at least one sublocation comprises at least two cell types selected from the group consisting of neoplastic cells, fibrous tissue, inflammatory tissue, necrotic cells and apoptotic cells. In another embodiment of the invention, each sublocation on the microarray comprising neoplastic cells comprises at least one of fibrous tissue, inflammatory tissue, necrotic cells and apoptotic cells. In a further embodiment, each sublocation comprises a mixture of both cancerous and non-cancerous or normal cells.

In still a further embodiment, sublocation tissues are selected from an oncology repository or a collection of tissue specimens that represent the most common neoplastic diseases. These include, but are not limited to:

| Specimen | Disease Type |
|---|---|
| prostate | BPH: adenocarcinoma |
| breast | benign and metastic, DCIS, stage 1-IV |
| colon | adenoma, adenocarcinoma |
| brain | cerebellular hemangiomas, gliomas |
| ovary | ovarian cancer |
| cervix | cervical cancer, dysplastic, CIN |
| lung | adenocarcinoma, small cell carcinoma |
| liver | hepatocellular carcinoma |
| bladder | transitional carcinoma |
| thyroid | medullary carcinoma |
| adrenal | adrenal carcinoma |
| parathyroid | parathyroid tumors |
| pancreas | pancreatic cancer, islet cell tumors |
| soft tissue | Ewing sarcoma |
| integumentary | malignant melanoma, benign nevi |

Oncology tissue microarrays are further described U.S. Provisional Application Serial No. ______ entitled “Oncology Tissue Microarrays,” filed Sep. 29, 2000 (Attoney Docket No. 5568/1040), the entirety of which is incorporated by reference herein.

In a further embodiment, microarrays are provided wherein the sublocations are selected from a repository of tissues representing diseases affecting women.

| Specimen | Disease Types |
|---|---|
| breast | pagets, cancer, benign disease |
| uterus | endometriosis |
| cervix | dysplasia, HPV, cancer |
| ovaries | diseased, including cancer |
| fallopian tubes | diseased, including cancer |

In another embodiment of the invention, sublocations are selected from a repository of endocrine tissue specimens:

| Specimen | Disease Types |
|---|---|
| thyroid | hyper- and hypo-thyroidism |
| Parathyroid | cancer, adenoma |
| Adrenals | adenoma, cancer |
| Pancreas | diabetes, islet cell tumors |
| Breast | hyperplasia, tumor |
| Ovary | benign cancer |
| Bone | estrogen replacement, osteoporosis |
| prostate | bph, cancer |

In one embodiment of the invention, cardiovascular and neurological tissues are selected from a repository of cardiovascular and neurological tissue specimens:

| Specimen | Tissue Sources/Disease Loci |
|---|---|
| heart | cardiac tissues, special dissections |
| vascular | vein, artery, aorta, others |
| brain | full brain, specific anatomical structures |
| spinal cord | whole cords, DRG, others |
| neural tissues | nerve tissues, neural tubes |

In one embodiment of the invention, tissues are selected from a repository of tissue specimens from individuals having an autoimmune disease:

| Specimen | Tissue Sources/Disease Loci |
|---|---|
| osteoporosis | bone, whole blood, lymphocytes, synovia |
| rheumatoid arthritis | bone, whole bloods, lymphocytes, synovia |
| osteoarthritis | bone, whole blood, lymphocytes, synovia |

In one embodiment of the invention, tissues are selected from a repository of tissue specimens from individuals having a respiratory disease:

| Specimen | Disease Types |
|---|---|
| lung | adenocarcinoma, adenoma |
| lung | autoimmune disease, asthma |
| bronchial washings | CIN |

In one embodiment of the invention, tissues are selected from a repository of tissue specimens from individuals having an infectious disease:

| Specimen | Tissue Sources/Disease Loci |
|---|---|
| HIV infection | all organs, blood, drug resistance |
| HCV infection | IV drug abuser population |
| HBV infection | Hospital acquired, IV drug user population |
| EBV infection | Epstein Barr |
| CMV infection | transplant patients |
| HPV infection | cervical, dermatological |

It should be obvious to those of skill in the art that the above tables represent non-limiting lists of the types of tissues that may be in a particular repository, and that other tissues belonging to the repository categories identified are encompassed within the scope of the invention. Nor does the term, “repository” imply that the tissue must be stored for any particular length of time. In one embodiment, tissue which is freshly obtained (e.g., processed within less than ten hours) from a surgical or other pathology procedure can be identified as belonging to a repository (e.g., by obtaining information relating to at least one biological characteristic, such as the characteristic that would qualify the tissue for placement in the repository, and recording this information).

In one embodiment, the microarray comprises at least one sublocation comprising a disease tissue selected from any of the repositories listed above and at least one sublocation comprising a normal tissue, either from the same individual from which the disease tissue is obtained, or from a normal specimen of the same tissue type as the diseased tissue, but from a different individual. In another embodiment of the invention, sets of sublocations (e.g., two or more) comprise tissues of the same type, but different disease stages. In a further embodiment, the microarray comprises tissues selected from the group consisting of tissues from a cancer tissue repository, tissues from a repository of tissues representing diseases affecting women, tissues from a repository of endocrine tissue specimens, tissues from a repository of cardiovascular tissues specimens, tissues from a repository of neurological tissue specimens, tissues from a repository of tissue specimens from individuals having an autoimmune disease, a respository of tissue specimens from individuals having a respiratory disease, and tissues from a repository of tissue specimens from individuals having an infectious disease.

In another embodiment of the invention, the sublocations comprise cells which represent the course of a progressive disease in a target tissue or organ (e.g., such as cancer, Parkinson's disease, muscular dystrophy, and the like) and the microarray further includes non target tissues or organs. For example, a Parkinson's disease array would comprise brain cells from individuals at various stages of the disease, and would also include any of: liver cells, muscle cells, skin cells, other types of cells and combinations thereof. In the case of a cancer progression microarray, in one embodiment, sublocations are provided which represent different grades of cancer in the primary tissue affected (e.g., breast tissue in the case of breast cancer). In another embodiment, sublocations are also provided which include lymphoid tissue, lung tissue, and tissues where there are secondary metastases. In a further embodiment, sublocations are provided which comprise normal cells which neighbor cancer cell(s) and/or normal cells from other tissues in the body.

In one embodiment of the invention, the microarray comprises a plurality of tissues representative of breast cancer progression, as shown in FIGS. 2A-2E, for example. In one embodiment of the invention, tissues at the different sublocations of the microarray are selected from the group consisting of normal breast tissue, ductal carcinoma in situ, invasive ductal breast cancer (grade 1), invasive ductal breast cancer (grade 2), invasive ductal breast cancer (grade 3), lymph node metastases from the same any of: ductal carcinoma in situ, invasive ductal breast cancer (grade 1), invasive ductal breast cancer (grade 2), and invasive ductal breast cancer (grade 3). In another embodiment of the invention, the microarray also comprises at least one control tissue selected from the group comprising of brain, heart, liver, spleen, muscle, lymph node, testis, kidney, thyroid, adrenal gland and prostate, but at least normal breast tissue. In a further embodiment of the invention, the sublocations represent Grade I T1N0M0, Grade II T2N1M0, Grade III T3N2M1, Grade IV T3N2M2 tissue samples, and/or Grade HER-2/neu/+, ER/PR+, and Breast ER/PR− tissue samples (grading according to the World Health Organization).

In a further embodiment, the second location comprises a microarray comprising a plurality of cells representative of disease progression in lung cancer. In one embodiment, the microarray comprises normal lung parenchyma, normal bronchi, different subtypes of adenocarcinoma, squamous cell carcinoma, undifferentiated large cell carcinoma, small cell carcinoma, as well as lymph node metastases from tumors included in the microarray (e.g., paired metastases). In a further embodiment, control sublocations are also provided comprising at least one of brain, heart, liver, spleen, muscle, lymph node, testis, kidney, thyroid, adrenal gland or prostate tissue.

In another embodiment, the microarray comprises a plurality of cells representative of disease progression in colorectal cancer. In one embodiment, the microarray comprises normal colon mucosa from patients having no history of colorectal cancer, paired tissues comprising normal and cancerous colon mucosa (e.g., from the same patient), adenoma with mild dysplasia, adenoma with severe dysplasia, nodal negative colorectal cancer, nodal positive colorectal cancer, and lymph node metastases from any of these cancerous cells. In a further embodiment, control sublocations are also provided comprising at least one of brain, heart, liver, spleen, muscle, lymph node, testis, kidney, thyroid, adrenal gland and prostate tissue. In another embodiment of the invention, the microarray comprises at least cells from tumors representing the 4 Dukes'stages: e.g., A, tumor within the intestinal mucosa; B, a tumor which has invaded into muscularis mucosa; C, metastasis to lymph nodes and D, metastasis to other tissues.

In still another embodiment, the microarray comprises a plurality of cells representative of disease progression in prostate cancer, including sublocations which represent Gleason Grades 1-10, or 1-6, and 4-10, or 6-10, and including control sublocations, comprising at least one of, brain, heart, liver, spleen, muscle, lymph node, testis, kidney, thyroid, adrenal gland and prostate tissue.

While in the embodiments described above tissues are preferably obtained from humans, in some embodiments, tissues are obtained from a non-human individual. In one embodiment, the cells at each sublocation are obtained from the tissues of a single type of adult non-human mammal, e.g., such as a rat, a rabbit, a mouse, a ferret, a non-human primate, a domestic animal (e.g., dogs, cats, cows, sheep, goats, pigs, and the like). In another embodiment of the invention, the array comprises cells from a non-mammalian organism, including yeast, hydra, dictyostelium, Arabidopsis, nematodes, fruitflies, frogs, fish (e.g., zebrafish), and sharks.

However, in another embodiment of the invention, the microarray comprises sublocations representing at least two different organisms, i.e., providing reagents to examine the expression of biological characteristics shared between different organisms (e.g., for examining the expression products of homeotic genes or other evolutionarily conserved genes). In one embodiment of the invention, the array comprises cells from at least two of: yeast, hydra, dictyostelium, Arabidopsis, nematodes, fruit flies, frogs, zebrafish, mice, rats, non-human primates, and humans.

In another embodiment, cells are obtained from tissues of a single organism at different stages of its development, e.g., such as yeast, hydra, dictyostelium, Arabidopsis, nematodes, fruit flies, frogs, zebrafish, mice, rats, non-human primates, and humans. In one embodiment of the invention, the cells are from different stages in the development of a single organ, such as the liver, brain, eye, skin, heart, lung, and spleen, reproductive organs, and thymus. In a further embodiment of the invention, the order of the sublocations on the array reflects a progression from the earliest stage of development to the latest stage of development represented by the sublocations on the array.

In still a further embodiment of the invention, the sublocations of the microarray comprise cells from genetically engineered organisms, organisms comprising naturally arising mutations, or genetically engineered cells (e.g., stably or transiently transfected cell lines, genetically engineered tumors, and the like). In one embodiment, the sublocations of the microarray comprise cells from any of: a knockout mouse (e.g., a p53 knockout, an MDM2 knockout, a VEGF knockout), a knock-in mouse, a transgenic mouse having an altered dose of a wild type gene, a modified form of a wild type gene, or a disrupted gene (comprising one or more point mutations, substitutions, deletions, translocations, and the like). In another embodiment, multiple organs or tissue types from a genetically engineered organism are represented on the microarray.

In one embodiment of the invention, the microarray comprises at least sublocations representing differing doses of the expression product(s) of a selected gene or biomolecule (e.g., DNA, mRNA, protein, or a modified, or processed form thereof). For example, in one embodiment, at least one sublocation comprises a tissue which does not express a selected gene, or expresses a less stable gene product, at least one sublocation comprises a tissue which does express the selected gene in normal amounts, while at least one other sublocation expresses multiple copies of the selected gene, or overexpresses a selected gene, or expresses a more stable gene product of the selected gene.

In one embodiment, the selected gene is a tumor suppressor gene. which prevents the establishment or growth of oncogenically transformed cells. A tumor suppressor gene can act, to halt or slow the proliferation of cells or, for example, by causing the cells to undergo apoptosis in response to a particular stimulus or condition. Examples of tumor suppressor genes include, but are not limited to, p57Kip2 (Matsuoka et al., 1995, Genes Dev. 9: 650-662; Lee et al., 1995, Genes Dev. 9: 639-649), p27^{kip1} (Polyak et al., 1994, Cell 78: 59-66; Polyak et al., 1994, Genes Dev. 8: 9-22; Toyoshima & Hunter, 1994, Cell 78: 67-73; Nobori et al., 1994, Nature 368: 753-756), p21 (WAF-1/CIP1; Steinman et al., 1994, Oncogene 9: 3389-3396; Waga et al., 1994, Nature 369: 574-578); Harper et al., 1993, Cell 75: 805-816; Harper et al., 1995, Mol. Bio. Cell 6: 387-400); El-Diery, 1993, Cell 75: 817-825; El-Diery, 1994, Cancer Res. 54: 1169-1174; Florenz-Rozas et al., 1994, Proc. Natl. Acad. Sci. U.S.A. 91: 8655-8659; Halvey et al., 1995, Science 267: 1018-1021; Jiang et al., 1994, Oncogene 9: 3389-3396; Michieli et al., 1994, Cancer Res. 54: 3391-3395; Parker et al., 1995, Science 267: 1024-1027; and Sheiki et al., 1994, Oncogene 9: 3407-3415); p15 (Hannon & Beach, 1994, Nature 371: 257-261; Jen et al., 1994, Cancer Res. 54: 6353-6358); p16 (Cairns et al., 1994, Science 265: 415-417; Jen et al., 1994, supra; Spruck et al., 1994, Nature 370: 183-184), p18 (Guan et al., 1994, Genes Dev. 8: 2939-2952), p53 (Dulic et al., 1994, Cell 76: 1013-1023; Martinez et al., 1991, Genes Dev. 5: 151-159), Rb (Weinberg, 1990, Trends Biochem Sci. 15:199-202) and p107 (Zhu et al., 1993, Genes Dev. 7: 1111-1125). See also: Hunter & Pines, 1994, Cell 79: 573-582; Draetta, 1993, Trends Cell. Biol. 3: 287-289; Seizinger et al., 1991, Cytogenet. Cell. Genet. 58: 1080-1096; Soloman, 1993, Curr. Opin. Cell Biol. 5: 180-186; and Elledge et al., 1994, Curr. Opin. Cell. Biol. 6: 847-852, which discuss tumor suppressors and cell cycle regulators. The entireties of these references are incorporated by reference herein.

Additional Features of the Substrate

In a further embodiment of the invention, the profile array substrate comprises a third location for placing an identifier (e.g., a wax pencil mark or crayon mark, an etched mark, a label, a bar code, a microchip for transmitting radio or electronic signals, and the like). In one embodiment, the third location comprises frosted glass (e.g., where the substrate is a glass slide). In one embodiment, the identifier which is provided at the third location is a microchip which communicates with a processor which comprises, or can access, stored information relating to the identity and address of sublocations on the array, and information including patient information, or information about the tissue source.

Construction of the Tissue Microarrays

Tissue microarrays according to the invention are generated by obtaining donor tissues from any of the tissue sources described above, embedding these tissues, and obtaining portions of the embedded tissue for placement in a “recipient block,” a block of embedding matrix which can subsequently be sectioned, each section being placed on any of the substrates described above. Therefore, in one embodiment, the invention encompasses recipient blocks for forming any of the microarrays disclosed above.

Embedding Tissues: Forming Donor Blocks

In one embodiment of the invention, tissues are obtained and either paraffin-embedded, plastic-embedded, or frozen. When paraffin-embedded tissues are used, a variety of tissue fixation techniques can be used. Examples of fixatives, include, but are not limited to, aldehyde fixatives such as formaldehyde, formalin or formol, glyoxal, glutaraldehyde, hydroxyadipaldehyde, crotonaldehyde, methacrolein, acetaldehyde, pyruvic aldehyde, malonaldehyde, malialdehyde, and succinaldehyde; chloral hydrate; diethylpyrocarbonate; alcohols such as methanol and ethanol; acetone; lead fixatives such as basic lead acetates and lead citrate; mercuric salts such as mercuric chloride; formaldehyde; dichromate fluids; chromates; picric acid, and heat.

Tissues are fixed until they are sufficiently hard to embed. The type of fixative employed will be determined by the type of molecular procedure being used, e.g., where the molecular characteristic(s) being examined include the expression of nucleic acids, isopentane, or PVA, or another alcohol-based fixative is preferred, paraffin is preferred for performing immunohistochemistry, in situ hybridization, and in general, for tissues which are going to be stored for long periods of time. When cells are obtained from plasma, the cells may be snap frozen. OCT embedding is optimal for morphological evaluations.

Embedding media encompassed within the scope of the invention, includes, but is not limited to paraffin or other waxes, plastic, gelatin, agar, polyethlene glycols, polyvinyl alcohol, celloidin, nitrocelluloses, methyl and butyl methacrylate resins or epoxy resins. Water-insoluble embedding media such as paraffin and nitrocellulose require that specimens be dehydrated in several changes of solvent, such as ethyl alcohol, acetone, xylene, toluene, benzene, petroleum, ether, chloroform, carbon tetrachloride, carbon bisulfide, and cedar oil. or isopropyl alcohol prior to immersion in a solvent in which the embedding medium is soluble. Water soluble embedding media such as polyvinyl alcohol, carbowax (polyethylene glycols), gelatin, and agar, can also be used.

In one embodiment, tissue specimens are freeze-dried by deep freezing in plastic tissue cassettes and storing them at −80-70° C., such as in liquid nitrogen. In one embodiment, the tissues are then covered with a cryogenic media, such as OCT, and kept at −80-70° C., until sectioned. Examples of embedding media for frozen tissues include, but are not limited to, OCT, Histoprep®′ TBS, CRYO-Gel®′ and gelatin. In another embodiment, a tissue freezing aerosol may be used to facilitate embedding of a donor frozen tissue block. An example of a freezing aerosol is tetrafluoroethane 2.2. Other methods known in the art may also be used to facilitate embedding of a tissue sample and are encompassed within the scope of the invention.

Forming the Recipient Block

In one embodiment, microarrays according to the invention are constructed by coring holes in a recipient block comprising an embedding substance (e.g., paraffin, plastic, or a cryogenic media) and placing a tissue sample from a donor block in a selected hole. Holes can be of any shape and size, but are preferably made in a regular pattern. In one embodiment of the invention, the hole for receiving the tissue sample is elongated in shape. In another embodiment, the hole is cylindrical in shape.

While the order of the donor tissues in the recipient block is not critical, in some embodiments, donor tissue samples are spatially organized. For example, in one embodiment, donor tissues represent different stages of disease, such as cancer, and are ordered from least progressive to most progressive (e.g., associated with the lowest survival rates). In another embodiment, tissue samples within a microarray will be ordered into groups which represent the patients from which the tissues are derived. For example, in one embodiment, the groupings are based on multiple patient parameters that can be reproducibly defined from the development of molecular disease profiles. In another embodiment, tissues are coded by genotype and/or phenotype. Tissue samples on the microarray can additionally be arranged according to treatment approach, treatment outcome, or prognosis, or according to any other scheme that facilitates the subsequent analysis of the samples and the data associated with them.

The recipient block can be prepared while tissue samples are being obtained from the donor block. However, in one embodiment, the recipient block is prepared prior to obtaining samples from the donor block, for example, by placing a fast-freezing, cryo-embedding matrix in a container and freezing the matrix so as to create a solid, frozen block. The embedding matrix can be frozen using a tissue freezing aerosol such as tetrafluorethane 2.2 or by any other methods known in the art. The holes for holding tissue samples can be produced by punching holes of substantially the same dimensions into the recipient block as those of the donor frozen tissue samples or slightly smaller holes, and discarding the extra embedding matrix.

Information regarding the coordinates of the hole into which a tissue sample is placed and the identity of the tissue sample at that hole is recorded, effectively addressing each sublocation on the microarray. In one embodiment of the invention, data relating to any, or all of, tissue type, stage of development or disease, tissue source, patient information, expression of biological characteristics and the like, is recorded and stored in a database, indexed according to the location of the tissue on the microarray. Data can be recorded at the same time that the microarray is formed, or prior to, or after, formation of the microarray.

The coring process can be automated using coring needles coupled to a motor, or some other source of electrical or mechanical power. In one embodiment of the invention, a microarray is generated using a Beecher Instruments Tissue Microarrayer (Beecher Instruments, Silver Springs, Md.), or an automated microarray as described in U.S. Pat. No. 6,103,518, the entirety of which is incorporated by reference herein. These devices basically consist of a turret containing two hollow core borer needles, one larger than the other, mounted on a platform with a spring mechanism. The smaller needle removes a core from the recipient block while a larger needle removes a core of tissue from the donor tissue block by means of stylet(s). The stylet is inserted into the smaller needle thereby injecting the donor tissue core into the hole made in the recipient block, while the same, or another, stylet is used to remove embedding media remaining in the smaller core borer needle, permitting its reuse. The stylets described in U.S. Pat. No. 6,103,518, are designed primarily for use with paraffin tissue sections. Stylets which are designed especially for use in arraying frozen tissues are described in U.S. patent application Ser. No. ______, filed Feb. 8, 2001, entitled, “Stylet for Use With Tissue Microarrayer and Mold, Attorney Docket No. 5568/1070 (the entireties of which are incorporated by reference herein).

In one embodiment of the invention, large format microarrays are provided which comprise at least one sublocation greater in at least one diameter than 0.6 mm. In another embodiment, at least one sublocation comprises a heterogenously expressed biomolecule which is expressed in less than 80% of cells in a given tissue type and which is diagnostic of a disease. In a further embodiment of the invention, a large format microarray is provided which comprises at least one sublocation comprising at least two different cell types or cellular material (e.g., any of abnormally proliferating cells (e.g., cancerous cells), stromal cells, extracellular matrix, necrotic cells and apoptotic cells).

Large format microarrays can be used alone or in conjunction with small format microarrays (microarrays in which individual sublocations are less or equal to 0.6 mm in diameter). In one embodiment of the invention, a large format microarray is used in conjunction with a small format microarray derived from the same patient's tissue sample. In this embodiment, the large format microarray can be used to demonstrate that the biological characteristics of the smaller sublocations of the small format microarray are representative of the biological characteristics within a larger sample. Methods of constructing large format microarrays are disclosed in U.S. patent application Ser. No. ______, filed Feb. 9, 2000, entitled, “Large Format Microarrays,” Attorney Docket No. 5568/1170, the entirety of which is incorporated by reference herein.

Other methods of generating microarrays are described in U.S. Provisional Application No. 60/213,321, and in WO 99/44062 and WO 99/44062, the entireties of which are incorporated entirely by reference herein, and are encompassed within the scope of the instant invention.

Methods of Using Profile Array Substrates Comprising Microarrays

High throughput gene discovery methods are generating massive amounts of data, and follow-up characterization is critical to validate the involvement at candidate drug targets (e.g., genes expressed in diseased tissues and involved in disease progression), avoiding unnecessary expenditures of time on failed targets, or missing the best targets.

The profile array substrates according to the invention array a plurality of different types of biomolecules (e.g., DNA, RNA, proteins, and modified, or processed forms thereof) for analysis on a single substrate by providing a microarray at a second location for comparison to a test tissue at a first location.

The profile array substrates according to the invention enable the user to carry out rapid, extensive, and highly systematic surveys of candidate drug targets against very large panels of highly-characterized tissue populations.

Applications for the profile array substrates according to the invention include:

performing comprehensive molecular profiling of large numbers of specimens

searching for diagnostic, prognostic and clinical correlations;

investigating disease pathogenesis and progression;

selecting promising drug targets;

sorting/prioritizing cDNA array data;

surveying entire populations; and

validating gene discoveries in hundreds of human tissue specimens.

Providing a control microarray on a profile array substrate provides a more accurate interpretation of results along with the quality assurance of laboratory competence. For example, a labeled antibody which is identified as being able to specifically detect breast cancers which are estrogen responsive should stain estrogen responsive breast cancer tissues on the microarray to the same degree, and should not label normal breast tissue or should label normal tissue at significantly reduced levels, as determined by routine statistical testing to within 95% confidence levels.

Using Profile Array Substrates in Molecular Profiling

In one embodiment, a test tissue sample is obtained from an individual suspected of having a trait, (e.g., a disease, such as cancer), and is placed on a profile array substrate at the first location. In one embodiment, the profile array substrate comprises at a second location, a microarray comprising a plurality of sublocations which each represent different stages in the progression of the disease. The test sample and the microarray are contacted with a molecular probe reactive with a biomolecule and the reactivity of the molecular probe is measured to provide an indicia of the presence, absence or form the biomolecule (e.s., modified or unmodified).

Reactivity can be any of: binding, cleavage, processing, and/or labeling, and the like, and reactivity of the molecular probe with the test tissue is compared with reactivity of the molecular probe with the different sublocations on the microarray. In one embodiment of the invention, reactivity of the sublocations on the microarray in at least one test is known and is characteristic of a biological trait, such that reactivity of the test tissue is indicative that the test tissue shares that biological trait.

In one embodiment of the invention, data relating to the reactivity of the test tissue and the sublocations of the microarray are entered into a database, and information relating to the biological trait is made accessible, along with other data relating to the tissues at each sublocation on the array, to the user.

In another embodiment of the invention, information relating to the individual from whom the test tissue was obtained is entered into the database (e.g., age, sex, weight, race, patient history, family history, drug treatment history, and other traits as discussed above). In another embodiment of the invention, the database represents information from a population of individuals. In a further embodiment of the invention, the individual from whom the test tissue is obtained has at least one trait in common with the population.

In one embodiment of the invention, reactivity of the molecular probe with different tissues is not known, and information relating to reactivity with the test tissue and the cells in different sublocations in the array is determined. Data relating to the reactivity of the test tissue, and/or microarray, and the traits of the tissue source of the test tissue are then entered into the database. In another embodiment of the invention, a test tissue is contacted with different distinguishable molecular probes (e.g., a fluorescent antibody specific for Her-2/neu and a rhodamine labeled antibody specific for PSA), and a plurality of different reactivities is determined, and entered into the database. In still another embodiment of the invention, sets of identical profile array slides (e.g., from the same recipient block) are assayed in parallel using multiple samples of the same test tissue (e.g., from neighboring sections of a test block of embedded test tissue), expanding the number of different molecular probes being tested against the test tissue. In this way, a molecular profile of the test tissue can be determined and compared with the molecular profile of the microarray tissues.

In a further embodiment, the profile array substrate communicates with a detector which displays an image of the test tissue sample, and/or the tissues on the microarray. In another embodiment of the invention, the detector further communicates with a processor which stores or can access information in the database.

In one embodiment, a user of a profile array substrate is provided with access to information regarding the microarray stored in a specimen-linked database which comprises records stored in any of visual, auditory, or electronic form. In one embodiment, as shown in FIG. 2D, information is in the form of printed information regarding the tissues on the microarray. In another embodiment of the invention, the user is provided with access to a tissue information system which comprises at least one user device connectable to a network, as described in copending application Ser. No. ______, filed Feb. 9, 2001, entitled, “Specimen-Linked Database,” (Attorney Docket No. 5568/1020), the entirety of which is incorporated by reference herein. In one embodiment, access is provided by providing the user with an identifier identifying the microarray and a password for accessing the system and allowing the user to enter the identifier on an interface displayed by the user devise. The user device (e.g., a computer) communicates the identifier to an electronic database (either stored within the memory of the user device or accessed through a server) which comprises information relating to the microarray identified by the identifier.

In one embodiment, the electronic database is coupled to an information management system comprising search and relationship-determining functions for searching and classifying information within the database. For example, in one embodiment, in response to a user query entered on an interface displayed by the user device, the information management system downloads records in the database relating to the particular tissues on the microarray and classifies them by type or attribute (e.g., patient sex, age, disease, exposure to drug, tissue type, cancer grade, and the like).

In one embodiment of the invention, the information management system identifies relationships between the biological characteristics of a test tissue and tissues on the microarray, or other previously characterized tissues for which information is stored in the electronic database. The tissue information system can analyze relationships between the stored data already in the electronic database and the data relating to the test tissue using any method standardly used in the art, including, but not limited to, regression, decision trees, neural networks, and fuzzy logic, and combinations thereof. Programs for identifying relationships between data sets are known in the art, and include the Spotfire™ program as described in U.S. Pat. No. 6,014,661, the entirety of which is incorporated by reference herein.

The user device then displays at least one relationship or identifies that no discernable relationship can be found. In one embodiment, the user device displays a plurality of relationships on an interface of the display of the user device and displays information relating to the statistical probability that the relationship exists. The user selects among a plurality of relationships identified by the processor by interfacing with the interface to determine those of interest (e.g., a relationship which is a disease correlation might be of interest while a relationship regarding hair color might not be). In one embodiment of the invention, rather than scanning the entire database, the tissue information system samples the electronic database randomly until at least one statistically satisfactory relationship is identified, with the user setting parameters for what is “statistically satisfactory.”

In one embodiment of the invention, the relationship of interest is used to provide a diagnosis of a disease. In another embodiment of the invention, the relationship is used to provide a prognosis. In still another embodiment, the information is used to predict the likelihood of disease recurrence. In a further embodiment of the invention, the processor accesses other databases which comprise information relating to medical treatment of a particular disease, for example, demographic information, relating to individuals who are the source of the tissue, and other information, to further define relationships between the biological characteristics of the test tissue and the tissues for which information exists in the database. In still a further embodiment of the invention, the relationship of interest is used to identify the biological role of an uncharacterized gene.

Molecular Probes

Antibodies for Detection of Biological Characteristics

Antibodies specific for a large number of known antigens are commercially available. Alternatively, or in the case where the expression characteristics of an uncharacterized biomolecule, such as a polypeptide, is to be analyzed, one of skill in the art can raise their own antibodies, using standard techniques.

In order to produce antibodies, various host animals are immunized by injection with the growth-related polypeptide or an antigenic fragment thereof. Useful animals include, but are not limited to rabbits, mice, rats, goats, and sheep. Adjuvants may be used to increase the immunological response to the antigen. Examples include, but are not limited to, Freund's adjuvant (complete and incomplete), mineral gels such as aluminum hydroxide, surface active substances such as lysolecithin, pluronic polyols, polyanions, peptides, oil emulsions, keyhole limpet hemocyanin, dinitrophenol, and adjuvants useful in humans, such as BCG (bacille Calmette-Guerin) and Corynebacterium parvum. These approaches will generate polyclonal antibodies.

Monoclonal antibodies specific for a polypeptide may be prepared using any technique that provides for the production of antibody molecules by continuous cell lines in culture. These include, but are not limited to, the hybridoma technique originally described by Kohler and Milstein, 1975, Nature 256:495-497, the human B-cell hybridoma technique (Kosbor et al., 1983, Immunology Today 4:72; Cote et al., 1983, Proc. Natl. Acad. Sci. U.S.A. 80:2026-2030) and the EBV-hybridoma technique (Cole et al., 1985, Monoclonal Antibodies and Cancer Therapy, Alan R. Liss, Inc., pp. 77-96). In addition, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce growth-related polypeptide-specific single chain antibodies. The entireties of these references are incorporated by reference herein.

Antibody fragments which contain specific binding sites of a growth-related polypeptide may be generated by known techniques. For example, such fragments include, but are not limited to, F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science 246:1275-1281) to allow rapid and easy identification of monoclonal Fab fragments with the desired specificity to a growth-related polypeptide. An advantage of cloned Fab fragment genes is that it is a straightforward process to generate fusion proteins with, for example, green fluorescent protein for labeling.

Antibodies, or fragments of antibodies may be used to quantitatively or qualitatively detect the presence of growth-related polypeptides or conserved variants or peptide fragments thereof. For example, immunofluorescence techniques employing a fluorescently labeled antibody coupled with light microscopic, or fluorimetric detection can be used.

Antibodies or antigen binding portions thereof may be employed histologically, as in immunohisto chemistry, immunofluorescence, immunoelectron microscopy, or an histological assays, for in situ detection of polypeptides or otherantigent-contaiing biomolucules.

Immunohistochemistry (IHC)

In situ detection of an antigen can be accomplished by contacting a test tissue and microarray on a profile array substrate with a labeled antibody that specifically binds the antigen. The antibody or antigen binding portion thereof is preferably applied by overlaying the labeled antibody or antigen binding portion onto the test tissue and microarray. Through the use of such a procedure, it is possible to determine not only the presence of the antigen but also its amount and its localization in a test tissue and in the plurality of sublocations within the microarray.

In one embodiment, antibodies are detectably labeled by linkage to an enzyme for use in an enzyme immunoassay (EIA) (Voller, 1978, Diagnostic Horizons 2:1-7, Microbiological Associates Quarterly Publication, Walkersville, Md.); Voller et al., 1978, J. Clin. Pathol. 31:507-520; Butler, 1981, Meth. Enzymol. 73:482-523; Maggio, E. (ed.), 1980, Enzyme Immunoassay, CRC Press, Boca Raton, Fla.,; Ishikawa et al., (eds.), 1981, Enzyme Immunoassay, Kgaku Shoin, Tokyo). The enzyme which is linked to the antibody will react with an appropriate substrate, preferably a chromogenic substrate, in such a manner as to produce a chemical moiety which is detectable, for example, by spectrophotometric, fluorimetric or visual means. Examples of enzymes useful in the methods of the invention include, but are not limited to peroxidase, alkaline phosphatase, and RTU AEC.

Detection of bound antibodies can alternatively be performed by radiolabeling antibodies and detecting the radiolabel. Following binding of the antibodies and washing, the samples may be processed for autoradiography to permit the detection of label on particular cells in the samples.

In one embodiment, antibodies are labeled with a fluorescent compound. When the fluorescently labeled antibody is exposed to light of the proper wavelength, its presence can be detected due to fluorescence. Many fluorescent labels are known in the art and may be used in the methods of the invention. Preferred fluorescent labels include fluorescein, amino coumarin acetic acid, tetramethylrhodamine isothiocyanate (TRITC), Texas Red, Cy3.0 and Cy5.0. Green fluorescent protein (GFP) is also useful for fluorescent labeling, and can be used to label non-antibody protein probes as well as antibodies or antigen binding fragments thereof by expression as fusion proteins. GFP-encoding vectors designed for the creation of fusion proteins are commercially available.

The primary antibody (the one specific for the antigen of interest) may alternatively be unlabeled, with detection based upon subsequent reaction of bound primary antibody with a detectably labeled secondary antibody specific for the primary antibody. Another alternative to labeling of the primary or secondary antibody is to label the antibody with one member of a specific binding pair. Following binding of the antibody-binding pair member complex to the sample, the other member of the specific binding pair, having a fluorescent or other label, is added. The interaction of the two partners of the specific binding pair results in binding the detectable label to the site of primary antibody binding, thereby allowing detection. Specific binding pairs useful in the methods of the invention include, for example, biotin:avidin. A related labeling and detection scheme is to label the primary antibody with another antigen, such as digoxigenin. Following binding of the antigen-labeled antibody to the sample, detectably labeled secondary antibody specific for the labeling antigen, for example, anti-digoxigenin antibody, is added which binds to the antigen-labeled antibody, permitting detection.

The staining of tissues for antibody detection is well known in the art, and can be performed with molecular probes including, but not limited to, AP-Labeled Affinity Purified Antibodies, FITC-Labeled Secondary Antibodies, Biotin-HRP Conjugate, Avidin-HRP Conjugate, Avidin-Colloidal Gold, Super-Low-Noise Avidin, Colloidal Gold, ABC Immu Detect, Lab Immunodetect, DAB Stain, ACE Stain, NI-DAB Stain, polyclonal secondary antibodies, biotinylated affinity purified antibodies, HRP-labeled affinity purified antibodies, and/or conjugated antibodies.

In one embodiment, immunohistochemistry is performed using an automated system such as the Ventana ES System and Ventana gen^{II} ™ System (Ventana Medical Systems, Inc., Tucson, Ariz.). Methods of using this system are described in U.S. Pat. Nos. 5,225,325, 5,232,664, 5,322,771, 5,418,138, and 5,432,056, the entireties of which are incorporated by reference herein.

IHC techniques used in conjunction with the profile array substrates according to the invention can provide a powerful diagnostic tool. For example, in one embodiment, profile array substrates are used to diagnose cancers which are otherwise not morphologically identifiable, such as small round blue cell tumors found in children. These tumors show no distinguishing morphological features but require positive identification because of they require specific therapies to treat. IHC has proven to be one of the most powerful diagnostic tools to help categorize these tumors. In the majority of cases, a carefully selected panel of antibodies can assist in identifying most of the small blue round tumors such as leukemia/lymphoma, Ewing's Sarcoma, rhabdomyosarcoma, and mesenchymal chrondrosarcoma. In one embodiment, a panel of antibodies is identified using molecular profiling techniques described above.

In many cancers, although staining by one specific antibody may not be diagnostic, a diagnostic pattern of staining by a combination of antibodies may be observed. Profile array substrates therefore offer a wide range of positive controls on a single slide that are useful for validating staining and antibody sensitivity of tissues to a combination of antibodies.

For example, in one embodiment, diagnostic markers for use in diagnosing breast cancer are selected from the group consisting of BRCA1, BRCA2, Her-2/neu, C-Met, GalNAc, MAGE-3, and CK20 gene mutations and/or gene products, and combinations thereof (see, e.g., U.S. Pat. Nos. 6,057,105 and 6,162,897, the entireties of which are incorporated herein), and the expression of at least one, at least two, at least three, or at least four of these markers is used to diagnose the presence or absence of cancer in a test sample of breast tissue, on one, or a plurality of, identical profile array substrates. In one embodiment, while the detection of any one marker may be diagnostic, the detection of multiple markers is used to enhance the sensitivity and specificity of the assay. In one embodiment, multiple markers are detected in a single assay of a profile array substrate, e.g., using differentially labeled molecular probes. However, in another embodiment, sets of identical profile array substrates comprising identical microarrays are evaluated using different molecular markers.

In a further embodiment, a profile array substrate is used to evaluate the progression of disease in a test tissue sample by providing a microarray at a second location comprising tissues representing different stages of the disease. In one embodiment, at least one biological characteristic of a test tissue sample is evaluated by IHC to determine the expression status of a biological characteristic associated with a particular stage of breast cancer, prostate cancer, colon cancer, skin cancer, lung cancer, and the like. In one embodiment, the disease being evaluated is breast cancer, and the biological characteristic is a breast-cancer specific marker which specifically identifies a particular stage of breast cancer. In another embodiment, the biological characteristic is any of Estrogen (ER) and Progesterone receptor (PR) status, expression of Ki67, c-erbB-2, nmt55, Her-2/neu, nm23, MLN 50, MLN 51, MLN 62 MLN 64, EGF-R, p53, or combinations thereof. In a preferred embodiment, prognostic information is preferably obtained by evaluating ER and PR status (e.g., using antibodies which specifically bind to these receptors).

In one embodiment, a microarray is provided comprising at least 20 different types of breast cancers (e.g., from 20 genetically unrelated individuals) for simultaneous testing with a test tissue to determine the presence or absence of a cancer-specific and/or breast cancer-specific marker(s) in the test tissue. The user is able to access information relating to sublocations of the microarray which show the same pattern of expression of marker(s) as the test sample because information relating to identical microarray(s) (tested using at least the same marker(s)) is stored in an information retrieval database to which the user has access. In one embodiment, in addition to information relating to the expression of cancer-specific and/or breast cancer-specific markers, the database comprises information relating to tumor size, node status, and metastasis of the breast cancer tissues provided on the microarray, as well as information relating to the particular patient(s) from whom the tissues were obtained.

Nucleic Acid Probes

Nucleic acid probes are also useful to correlate the differential expression of genes with particular traits (e.g., such as cancer or other diseases). In one embodiment, the sequence of a gene which is known to be associated with disease is used to generate a probe or primer for use in the present invention. Means for detecting specific DNA sequences within genes are well known to those of skill in the art. In one embodiment, oligonucleotide probes chosen to be complementary to a selected subsequence within the gene can be used.

Methods of labeling nucleic acids are well known to those of skill in the art. Preferred labels are those that are suitable for use in in situ hybridization (ISH) or fluorescent in situ hybridization (FISH). In one embodiment, nucleic acid probes are detectably labeled prior to hybridization with a tissue sample. Alternatively, a detectable label which binds to the hybridization product can be used. Labels for nucleic acid probes include any composition detectable by spectroscopic, photochemical, biochemical, immunochemical, or chemical means and include, but are not limited to, radioactive labels (e.g. ³²P, ¹²⁵I, ¹⁴C, ³H, and 35S), fluorescent dyes (e.g. fluorescein, rhodamine, Texas Red, etc.), electron-dense reagents (e.g. gold), enzymes (as commonly used in an ELISA), colorimetric labels (e.g. colloidal gold), magnetic labels (e.g. Dynabeads TM), and the like. Examples of labels which are not directly detected but are detected through the use of directly detectable label include biotin and dioxigenin as well as haptens and proteins for which labeled antisera or monoclonal antibodies are available.

A direct labeled probe, as used herein, is a probe to which a detectable label is attached. Because the direct label is already attached to the probe, no subsequent steps are required to associate the probe with the detectable label. In contrast, an indirect labeled probe is one which bears a moiety to which a detectable label is subsequently bound, typically after the probe is hybridized with the target nucleic acid.

Labels can be coupled to nucleic acid probes in a variety of means known to those of skill in the art. In some embodiments the nucleic acid probes are labeled using nick translation or random primer extension (Rigby, et a!. J. Mol. Biol., 113: 237 (1977) or Sambrook et al., Molecular Cloning: A Laboratory Manual, Cold Spring Harbor Laboratory, Cold Spring Harbor, N.Y., (1989), the entireties of which are incorporated by reference herein).

Alternatively, sequences or subsequences of tissues within a microarray may be amplified by a variety of DNA amplification techniques (e.g., polymerase chain reaction, ligase chain reaction, transcription amplification, etc.) prior to detection using a probe. Amplification of nucleic acid sequences increases sensitivity by providing more copies of possible target subsequences. In addition, by using labeled primers in the amplification process, the sequences are labeled as they are amplified.

In situ hybridization (ISH) and Fluorescent In Situ Hybridization (FISH)

In situ hybridization (ISH) and Fluorescent In Situ Hybridization (FISH) are techniques that can avail themselves to paraffin-embedded sectioned tissue. Both techniques are genomic based rather than proteomic based, as in IHC, and involve RNA and DNA probes that will hybridize, or specifically bind to their complement base sequence. In some embodiments, labels are attached to genomic probes that allow hybridization of the probes to be visualized under a microscope. ISH probes generally have a chromogenic marker and can be observed by traditional light microscopy. FISH probes generally have a fluorescent marker bonded and must be visualized with the use of a fluorescent microscope.

Although IHC markers are most useful in characterizing and identifying tumors, there are some lesions such as endocrine/neuroendocrine tumors in which ISH can add another level of specificity. This is because some tumors may take up proteins nonspecifically or may not be found in levels detectable by IHC. In these instances, genomic probes can be amplified and are more readily detectable. In breast cancer, FISH for the detection of cerbB-2 is commonly used for its strong predictive power (see, e.g., Kallioniemi, et al, PNAS 89: 5321-5325, 1992, the entirety of which is incorporated by reference herein).

In one embodiment, profile array substrates are used as control tools in ISH/FISH methodologies. Just as in IHC, the option of having a plurality of individual control specimens simultaneously processed with a test tissue is an extremely valuable tool for assaying probe sensitivity and detection.

In one embodiment, for in situ hybridization of paraffin-embedded tissues, sections of paraffin-embedded tissue immobilized on glass substrates are treated as follows: substrates are dewaxed in staining dishes by three changes in xylene for 2 minutes each (dewaxing is not necessary for non-embedded single cells); dewaxed samples are then rehydrated using the following procedure: exposure to 100% ethanol, two times for two minutes, then subsequent 2 minute incubations in 95%, 70%, and 50% ethanol. (It should be apparent to those of ordinary skill in the art that the incubation time is not critical and may be optimized, but in general should be at least two minutes.)

Samples are denatured (e.g., by incubation for 20 minutes at room temperature in 0.2 N HCl, followed by heat denaturation for 15 minutes at 70° C. in 2× SSC). Samples are then rinsed, for example, in 1× PBS for 2 minutes. In some situations, usually empirically determined, a pronase digestion step may be included here which later allows improved access of the probes to the nucleic acids contained within the tissue sections. In such cases, samples are digested for 15 minutes at 37° C. with pre-digested, lyophilized pronase at an empirically determined concentration which allows hybridization yet preserves the cellular morphology (e.g., such as 0.1 to 10 μg/ml).

Pronase-digested samples are incubated for 30 seconds in a wash buffer, such as 2 mg/ml glycine in 1× PBS, to stop the digestion process. Samples may be post-fixed, for example, using freshly prepared 4% paraformaldehyde in 1× PBS, for 5 minutes at room temperature. Fixation is stopped by further washes, e.g., a 5 minute incubation in 3× PBS, followed by two 30 second rinses in 1× PBS. Samples are then soaked in 10 mM DTT, 1× PBS, for 10 minutes at 45° C., followed by a 2 minute incubation in 0.1 M triethanolamine, pH 8.0 (triethanolamine buffer). Next, samples are placed in fresh triethanolamine buffer to which acetic anhydride is added to 0.25% final concentration, followed by mixing and 5 minutes' incubation with gentle agitation. In one embodiment, more acetic anhydride is added to a final concentration of 0.5%, followed by 5 minutes' further incubation. Samples are washed, for example, for 5 minutes in 2× SSC, and by dehydrated by successive incubation in 50%, 70%, 95% and 100% ethanol for 2 minutes each at room temperature. Preferably, samples are air-dried or dried with desiccant before proceeding to the hybridization step. Any, or all, of the preceding series of steps may be automated in order to increase throughput.

Probes for in situ hybridization may be DNA or RNA oligonucleotides (e.g., RNA transcribed in vitro). In one embodiment, RNA probes labeled with ³⁵S are dissolved in 50 mM dithiothreitol (DTT) and are added to a non-specific competitor. In one embodiment, the competitor is preferrably RNA made in the same manner as the labeled specific probe, except from a transcription template with non-specific sequences, such as a vector with no insert. No labeled ribonucleosides are in the reaction mix.

The probe/non-specific competitor mixture is then denatured, for example, by heating at 100° C. for 3 minutes, and added to a hybridization buffer (e.g., such as 50% (v/v) deionized formamide, 0.3 M NaCl, 10 mM Tris (pH 8.0), 1 mM EDTA, 1× Denhardt's solution, 500 mg/ml yeast tRNA, 500 mg/ml poly(A), 50 mM DTT, and 10% polyethylene glycol 6000) to a 0.3 μg/ml-10 μg/ml final probe concentration. An estimate of the amount of probe synthesized is based on a calculation of the percent of the label incorporated and the proportion of the labeled base in the probe molecule as a whole. In one embodiment, the non-specific competitor is provided in an amount approximately equal to one half the mass of labeled probe.

The probe/hybridization mix is incubated at 45° C. until applied to the microarrays and test tissue sample as a thin layer of liquid. Hybridization reactions are generally incubated in a moist chamber such as a closed container containing towels moistened with 50% deionized formamide, 0.3 M NaCl, 10 mM Tris (pH 8.0), 1 mM EDTA, at 45° C. If background (e.g., the amount of non-specific labeling) proves to be a problem, a 1 to 2 hour pre-hybridization step using only non-specific, unlabeled riboprobe competitor in hybridization buffer can be added prior to the step in which labeled probe is applied.

In one embodiment, hybridization is carried out for 30 minutes to 4 hours, followed by washing to remove any unbound probe. In one embodiment, the profile array substrates are washed in an excess (100 ml each wash) of the following buffers: 50% formamide, 2× SSC, 20 mM β-mercaptoethanol, two times, for 15 minutes at 55° C.; 50% formamide, 2× SSC, 20 mM β-mercaptoethanol, 0.5% Triton X-100, two times, for 15 minutes at 55° C.; and 2× SSC, 20 mM β-mercaptoethanol, two times, for 2 minutes at 50° C.

In another embodiment, samples are subjected to RNAse digestion for 15 minutes at room temperature for example using a solution containing 40 mg/ml RNase A, 2 mg/ml RNase T1, 10 mM Tris (pH 7.5), 5 mM EDTA and 0.3 M NaCl. In one ebmodiment, after RNase digestion, slides are soaked two times for 30 minutes each in 2× SSC, 20 mM β-mercaptoethanol at 50° C., followed by two washes in 50% formamide, 2× SSC, 20 mM β-mercaptoethanol at 50° C. and two washes of 5 minutes each in 2× SSC at room temperature. Hybridized, washed slides are dehydrated through successive two minute incubations in the following: 50% ethanol, 0.3 M ammonium acetate; 70% ethanol, 0.3 M ammonium acetate; 95% ethanol, 0.3 M ammonium acetate; 100% ethanol. Slides are air dried overnight and with emulsion for autoradiography according to standard methods.

Sections prepared from frozen tissues may be hybridized by a similar method except that the dewaxing and paraformaldehyde fixation steps are omitted. For details, see Ausubel et al., 1992, Short Protocols in Molecular Biology, (John Wiley and Sons, Inc.), pp. 14-15 to 14-16, the entirety of which is incorporated by reference herein.

In a further embodiment of the invention, ISH or FISH probes or other nucleic acid molecular probes (e.g., DAPI, acridine orange) are used to evaluate the absolute amounts of nucleic acids in cells within a tissue (e.g., to determine the copy number of nucleic acids on the tissue) since changes in copy number of nucleic acids are often associated with the development of pathology. However, in another embodiment, ISH or FISH probes are used to detect allelic variants which result from mutations such as point mutations, deletions, insertions, or rearrangements in a gene of interest. In still another embodiment, ISH or FISH is performed with one or more amplification steps, i.e., such as by performing in situ PCR. A detailed description of these techniques are presented in Ausubel, et al., 1992, supra, pp. 14-37 to 14-49, the contents of which are hereby incorporated by reference.

In a further embodiment of the invention, information obtained from a single sublocation on a microarray can be information relative to the expression of both proteins and nucleic acids. For example, in one embodiment of the invention, after performing immunohistochemistry on tissue at a sublocation, a portion of the tissue is obtained to isolate nucleic acids which are further analyzed by amplification methods such as PCR. Detection of nucleic acids isolated from an embedded tissue sample is known in the art and is described in, for example, U.S. Pat. No. 6,013,461, U.S. Pat. No. 6,110,902, and U.S. Pat. No. 6,114, 110, the entireties of which are incorporated by reference herein.

In still a further embodiment, tissues can be counterstained to highlight their morphology (e.g., with hematoxylin/eosin, or another dye or combination of dyes, such as described in Ausubel et al., 1992, supra, pp. 14-19 to 14-22).

As with the IHC techniques described above, nucleic acid hybridization techniques can also be automated. In one embodiment, both detection and probing is automated. For example, in one embodiment, a profile array substrate which has been, or is being reacted, with a molecular probe is in communication with a detector. A light source in proximity to the tissue samples on the substrate transmits light to the samples and light transmitted by the samples is received by the detector. In one embodiment, the detector is in communication with the tissue information system described above and signals transmitted to the tissue information system relating to optical information from the tissues are displayed and/or stored within the electronic database. In one embodiment, optical information from tissue samples on the microarray is displayed as an image of tissue(s) on the interface of the display of a user device included in the tissue information system.

Selecting Promising Gene Drug Targets

In one embodiment, the molecular probe is a test probe and is used to identify a candidate drug target. Test probes can include antibodies, nucleic acids, enzymes, substrates, and the like, and are identified by screening any, or all of, a nucleic acid array (e.g., oligonucleotide arrays, cDNA arrays, Expressed Sequence Tag Arrays), a peptide array, a small molecule array, an array of cultured cells (primary or continuous cultures), or a tissue microarray.

Test probes are used to identify a biomolecule, or set of biomolecules, whose expression is diagnostic of a trait (e.g., such as by using the molecular profiling techniques described above). In one embodiment, identifying diagnostic biomolecules is performed by determining which molecules on a microarray are always present in a disease sample, and always absent in a healthy sample, or always absent in a disease sample and always present in a healthy sample, or always present in a certain form or amount in a disease sample and always present in a certain other form or amount in a healthy sample.

Test probes identifying diagnostic biomolecules are then contacted with a profile array substrate according to the invention to identify the presence, amount, and/or form of the diagnostic biomolecules in a microarray comprising different types of healthy and/or diseased tissues. In this way, a correlation between the expression of the diagnostic biomolecule(s) and a disease state may be validated. In another embodiment, a microarray is assayed along with a test tissue sample placed at a first location on the profile substrate array and validation is performed at the same time that a test tissue sample is evaluated for the presence, amount, and/or form of diagnostic biomolecules to obtain diagnostic information relating to the test sample.

In another embodiment of the invention, the expression of the diagnostic molecule is examined in a microarray comprising tissues from a drug-treated patient and tissues from an untreated diseased patient and/or from a healthy patient. In this embodiment, the efficacy of the drug is monitored by determining whether the expression profile of the diagnostic(s) molecule returns to a profile which is substantially similar (e.g., not significantly different as determined by routine statistical testing) from that a healthy patient. In one embodiment of the invention, the test tissue at the second location is obtained from a patient treated with a drug, while the microarray comprises at least both disease tissue (e.g., tissue comprising cells which are the target of disease) and healthy tissue of the same tissue type as the test tissue, and the expression of the diagnostic molecule(s) in the test tissue is compared with the expression pattern of the diagnostic molecule in the diseased or healthy tissue. In this embodiment, a drug is identified as useful for further testing when the expression pattern in the test tissue is substantially the same as the expression pattern within the healthy tissue (to within 95% confidence levels).

In a further embodiment of the invention, the role of the diagnostic molecule(s) are evaluated by comparing the expression of the molecule(s) in different sublocations on the microarray(s) with information in a database relating to the type of tissue, its developmental stage, and/or to traits of the individual(s) from which the tissue is obtained (e.g., such as patient information).

Batch Control of Molecular Probes

The profile array substrates according to the invention provide multiple control samples for simultaneous staining alongside a test tissue to provide an assessment of the sensitivity and specificity of a molecular probe, such as an antibody. Instead of reacting one tissue as a known positive control, the substrates provide the option of reacting, in one embodiment, 25 controls all on the same slide. In one embodiment, the control tissue samples are already known to be positive or negative for the most common clinical molecular probes (e.g., antibodies) used.

In one embodiment of the invention, profile array substrates are provided comprising tumor specific microarrays. For example, in one embodiment, a breast cancer microarray is provided which comprises at least 20 sublocations of different breast cancers and 1-5 sublocations of normal breast tissue. In one embodiment, a molecular probe or panel of molecular probes used to test the microarray has different known reactivities with a molecular probe, such as staining qualities (e.g., amount of staining and/or location of staining) among each of the sublocations. For example, in one embodiment, two common breast cancer marker antibodies, such as Estrogen receptor and c-erbB-2 are used to stain different breast cancer sublocations in the microarray. By comparing the staining quality, location and intensity of antibody staining of tissue samples at different sublocations of the microarray and an identical substrate comprising prestained sublocations, the ultimate in quality control can be achieved.

Routine Histology Lab Quality Control: Automated or Manual Methods IHC

The profile array substrates according to the invention can also be used in daily quality control for immunohistochemistry, or in other procedures that rely on molecular probes (e.g., nucleic acid hybridizations, and the like). A normal tissue microarray comprising a plurality of different non-diseased tissues is ideally suited for this purpose. By comparing substrate slides stained daily with previous daily runs that have been judged to have “optimal reactivity,” deviations in specificity and sensitivity in a molecular probe can be observed and corrections made. In one embodiment of the invention, comparing is done visually.

In another embodiment of the invention, comparing is done by collecting optical data (e.g., spectral data) from labeled molecular probes using an optical system in communication with the profile array substrate. In this embodiment, quantitative measurements are obtained. In another embodiment of the invention, the quantitative measurements so obtained are compared to measurements identified as optimal, and an antibody is identified whose optical measurements are substantially similar to those of the optimal measurement (as determined by routine statistical analysis with confidence levels set at 95%), as one to use in further tests. In another embodiment of the invention, the batch testing and identification of the antibody is automated. It should be obvious to those of ordinary skill in the art that batch testing can be done of any reactive molecular probes (e.g., a nucleic acid probe or enzyme probe).

Screening of New, Commercial Antibodies: Automated or Manual Methods

All newly acquired lots of commercial antibodies should be tested prior to diagnostic use for optimal titration and staining specificity. This can easily be accomplished with the use of the appropriate profile array substrate for the specific antibody being tested. For example, in one embodiment, a new batch of PSA antibody is evaluated on at least 5-100 individual prostate tumors by using a prostate microarray as described above.

Antibody Characterization: Automated or Manual Methods

An antibody characterization study involves the targeting of a supplied antibody to those tissues where gene expression is thought to occur. Substrates comprising normal tissue microarrays can be used to test for gene expression by providing a plurality of different normal tissues, while a substrate comprising a specific tumor microarray, such as a colorectal tumor microarray will be able to target the antibody to a purality of different colorectal tumors (e.g., from genetically unrelated individuals. In one embodiment, gene expression in various types and grades of a specific tumor as observed on a tumor microarray, such as a colorectal tumor microarray, is used to identify additional antibodies for use as molecular probes.

In one embodiment of the invention, at least one profile array substrate is provided which comprises an identical microarray (e.g., from the same recipient block, and preferably from neighboring sections within the same recipient block) as the one being tested and which is prestained for any of the following markers, Actin, CEA, Chromogranin, Cytokeratin, Desmin, EMA, GFAP, HMB, MSE, PLAP, PSA & PSAP and Vimentin, to provide additional controls.

Kits

Kits are contemplated for use in the methods according to the invention, comprising any of the profile array substrates described above and including a means to access information about the microarray, the information including, but not limited to, patient information and information about the biological characteristics of the tissue. In one embodiment of the invention, a kit includes any or all of a breast tissue microarray comprising at least 20 sublocations comprising different breast tumor types, and including 5 normal tissue sublocations and including at least one normal breast tissue sample; a colon cancer microarray comprising at least 20 sublocations comprising different colon tumor types, and including 5 normal tissue sublocations and including at least one normal colon tissue sample; a prostate cancer microarray comprising at least 20 sublocations comprising different prostate tumor types, and including 5 normal tissue sublocations including at least one normal prostate tissue sample; a lung cancer microarray, comprising at least 20 sublocations comprising different lung cancer tumor types and including 5 normal sublocations including at least one normal lung tissue sample; and tumor microarray comprising different tumors obtained from different tumor tissue types, and including at least one normal tissue sublocation. It should be obvious to those of skill in the art that the numbers and types of sublocation on the microarray of the profile array substrate can vary, and that these are merely exemplary embodiments.

In one embodiment, low density tissue microarrays are provided which comprise over 45-60 but less than 200 sublocations per slide. In one embodiment, the microarray comprises normal tissue and includes tissue samples from liver, lymph node, kidney, thyroid and prostate tissue. In a further embodiment, a kit is provided comprising high density tissue microarrays (over 200 tissue samples) which represent tissue samples from population surveys of both normal individuals and individuals with clinical conditions for the evaluation of gene expression patterns.

Kits may also include profile array substrates comprising microarrays comprising non-human tissue samples, such as microarrays including tissues from knockout animals comprising p53 disruptions. In one embodiment, the kit comprises at least one profile array substrate comprising a stained tissue (e.g., hematoxylin or eosin stained).

In a further embodiment of the invention, a kit is provided comprising control profile array substrates comprising tissue samples known to express a variety of antigens, e.g., in sets of 5 and 20 unstained slides. In one embodiment, each set further includes a stained substrate demonstrating positive staining of a specific antigen, and access to a technical description of the tissue staining characteristics of the microarray on the profile array substrate. Each set is labeled as to the tissue used and antigen identified by a particular staining pattern, or alternatively each set is labeled with an identifier which is cross-referenced with information stored in a database relating to the tissue used and antigen identified by a particular staining pattern.

In one embodiment, the following antigen/tissue combinations (e.g., antigens and tissues expressing particular antigens) are provided: Actin, normal colon; Actin, uterine smooth muscle; Carcinoembryonic antigen (CEA), colon adenocarcinoma; CD3, T-cell, tonsil; CD15/LeuM1, lymphoma; CD20/L26, B-cell, tonsil; CD30/BerH2, lymphoma; CD34, hematopoietic progenitor cells, tonsil; CD34, hematopoietic progenitor cells, normal skin; CD45/LCA, T-cell, tonsil; CD68/KPI, macrophage, tonsil; Chromogranin A, pancreas; Chromogranin A, pancreas slides; Cytokeratin, pan-keratin, normal prostate; Cytokeratin, pan-keratin, normal skin; Cytokeratin 7, breast ductal carcinoma; Cytokeratin 20, colon adenocarcinoma; Cytokeratin 20, bladder carcinoma; and Cytokeratin, high molecular weight, skin; Cytokeratin, high molecular weight, prostate; Desmin, leiomyoma; Desmin, normal colon; Epithelial membrane antigen (EMA), meningioma; Epithelial membrane antigen (EMA), breast; Glial fibrillary acidic protein (GFAP), brain; HMB45, melanosome, melanoma; HMB45, melanosome, melanoma, Clark score 1-5 w/nevus; Kappa light chains, tonsil; Lambda light chains, tonsil; Neuron specific enolase (NSE), pancreas; Placental alkaline phosphotase (PLAP), seminoma; Prostate specific antigen (PSA), prostate; 1132-5 Prostatic acid phosphotase (PSAP), prostate; S 100, skin; S 100, melanoma; Vimentin, tonsil; Vimentin, normal colon;Von Willebrand factor (Factor VIII), tonsil; Estrogen receptor, breast carcinoma; Progesterone receptor, breast carcinoma. In one embodiment the tissues are stanined by antibodies specific for the antigens.

Additional reagents and kit components include, but are not limited to antibodies, labels, DNA or RNA probes, and the like. In a further embodiment of the invention, the kit can comprise genomic DNA, RNA, cDNAs, oligonucleotides, from any or all of bladder, brain, breast, cervix, colon, esophagus, heart, small intestine kidney, liver, lung, skeletal muscle, pancreas, prostate, rectum, skin, spleen, stomach, and testis. The kit can further comprise buffers and other reagents for use in molecular procedures such as IHC, ISH, FISH, PCR, RT-PCR, and the like.

Variations, modifications, and other implementations of what is described herein will occur to those of ordinary skill in the art without departing from the spirit and scope of the invention as claimed. Accordingly, the invention is to be defined not by the preceding illustrative description but instead by the spirit and scope of the following claims.

What is claimed is:

1. A profile array substrate comprising a first location for placing a tissue sample and a second location comprising a microarray.

2. The profile array substrate of claim 1, wherein said microarray comprises a plurality of sublocations, each sublocation comprising a control tissue sample for which at least one biological characteristic is known.

3. A recipient block for forming the microarray of claim 1 or 2, wherein said recipient block comprises a frozen embedding media.

4. A method of evaluating a test tissue sample, comprising:
providing the profile array substrate of claim 1,
placing a test tissue sample at said first location;
reacting said test tissue and said microarray with a molecular probe,
comparing the reactivity of the test tissue with the reactivity of tissue samples in said microarray.

5. The method of claim 4, wherein said evaluating comprises determining the presence or absence of a pathology in said test tissue, and said microarray comprises at least one sublocation comprising a tissue sample having said pathology, and at least one sublocation comprising a tissue sample lacking said pathology.

6. The method of claim 5, wherein said determining is used to provide a diagnosis to an individual who is the source of said test tissue.

7. The method according to claim 5, wherein said microarray comprises a plurality of tissue samples representing different stages of a progressive disease.

8. The method according to claim 7, wherein said progressive disease is cancer.

9. The method according to claim 7, wherein said progressive disease is Parkinson's disease.

10. The method of claim 5, wherein information relating to the reactivity of said test tissue is stored in a database.

11. The method of claim 5, wherein the reactivity of tissue samples in the microarray to said molecular probe is known.

12. The method of claim 5, wherein the reactivity of tissue samples in the microarray to said molecular probe is unknown.

13. The method of claims 11 or 12, wherein information relating to the reactivity of tissue samples in the microarray is stored in a database.

14. The method according to claim 13, further comprising providing access to the database.

15. The method according to claim 13, wherein said database is an electronic database.

16. The method according to claim 15, wherein information relating to the reactivity of tissue samples in said microarray is compared to information relating to the source of the tissues.

17. The method according to claim 6, wherein said information relating to the source of the tissues is patient information.

18. The method according to claim 15, wherein said electronic database is coupled to an information management system.

19. The method according to claim 18, wherein said information management system comprises a search function and a relationship determining function.

20. The method according to claim 19, wherein said tissue information management system identifies relationships between the biological characteristics of the test tissue and the tissues of the microarray.

21. The method according to claim 20, wherein said relationship is the correlation of the expression of at least one biological characteristic of a tissue with the presence absence or stage of a disease.

22. The method according to claim 5, further comprising placing said profile array substrate in proximity to a light source, and transmitting light from said light source to at least one tissue on said substrate.

23. The method according to claim 22, further comprising providing a detector in proximity to said profile array substrate, said detector detecting light transmitted from said tissue.

24. The method according to claim 23, wherein said detector is in communication with a tissue information system comprising at least one user device connectable to the network, and wherein said user device is in communication with an electronic database comprising information relating to said tissues on said microarray.

25. The method according to claim 24, wherein said user device is capable of displaying an image of said tissue on a display of said user device.

26. A kit comprising at least one profile array substrate according to claim 1 or 2, and a means for accessing information relating to the sources of tissue on the microarray.

27. The kit according to claim 26, wherein said means for accessing information comprises an identifier identifying the microarray and a password for accessing an electronic database in which said information is stored.

28. The kit according to claim 26, further comprising at least one molecular probe.
